(19) United States
(12) Patent Application Publication
(10) Pub. No.: US 2025/0368600 A1
(43) Pub. Date: Dec. 4, 2025

(54) USE OF CYCLOHOMOGERANATE ESTERS AS AROMA CHEMICALS

(71) Applicants: BASF SE, Ludwigshafen am Rhein (DE); Studiengesellschaft Kohle gGmbH, Mülheim an der Ruhr (DE)

(72) Inventors: Mathias SCHELWIES, Ludwigshafen am Rhein (DE); Bernhard BRUNNER, Ludwigshafen am Rhein (DE); Ralf PELZER, Ludwigshafen am Rhein (DE); Florian GARLICHS, Ludwigshafen am Rhein (DE); Miriam BRU ROIG, Ludwigshafen am Rhein (DE); Benjamin LIST, Mülheim an der Ruhr (DE); Mathias TURBERG, Muelheim an der Ruhr (DE)

(21) Appl. No.: 18/876,688

(22) PCT Filed: Jun. 21, 2023
(86) PCT No.: PCT/EP2023/066754

§ 371 Date: Dec. 19, 2024

(30) Foreign Application Priority Data
Jun. 22, 2022 (EP) 22180423.0

(51) Int. Cl. C07C 69/608; C07C 67/333

(52) U.S. Cl. CPC C07C 69/608; C07C 67/333

(57) ABSTRACT

The present invention relates to the use of compound of formula (I) as an aroma chemical to impart a woody note, fruity note, herbal note, green note, rooty note, citrus note, petitgrain note, floral note, damascone note, tobacco note, ashtray note, gourmand note, sweet note, vetiver note, ionone note, ambra note, fresh note, natural note, fir needle note, camphoraceous note, or any combination of two or more of these impressions to composition and also for enhancing and/or modifying the aroma of a composition. The present invention is further directed to a composition comprising at least one compound of formula (I) and (i) at least one aroma chemical different from the compound of formula (I) or (ii) at least one non-aroma chemical carrier, or (iii) both (i) and (ii).

### FIELD OF INVENTION

The present invention relates to the use of compound of formula (I) as an aroma chemical to impart a woody note, fruity note, herbal note, green note, rooty note, citrus note, petitgrain note, floral note, damascone note, tobacco note, ashtray note, gourmand note, sweet note, vetiver note, ionone note, ambra note, fresh note, natural note, fir needle note, camphoraceous note, or any combination of two or more of these impressions to a composition and also for enhancing and/or modifying the aroma of a composition. The present invention is further directed to a composition comprising at least one compound of formula (I) and (i) at least one aroma chemical different from the compound of formula (I) or (ii) at least one non-aroma chemical carrier, or (iii) both (i) and (ii).

### BACKGROUND OF THE INVENTION

Aroma chemicals, especially fragrances, are of great interest, especially in the field of cosmetics, cleaning, and laundry compositions.

Despite a large number of already existing synthetic aroma chemicals, there is a constant need for new components in order to be able to satisfy the multitude of properties desired for extremely diverse areas of application. These include, firstly, the sensory properties, i.e. the compounds should have advantageous olfactory properties. Furthermore, aroma chemicals should also have additional positive secondary properties, such as e.g. an efficient preparation method, the possibility of providing better sensory profiles as a result of synergistic effects with other aroma chemicals, a higher stability under certain application conditions, a better higher substantivity.

Such properties are of special interest for compositions such as for example care compositions, hygiene articles, cleaning compositions, textile detergent compositions and compositions for scent dispensers.

Of special interest are aroma chemicals, which can impart one or more distinct sensory impressions to a composition, thereby contributing to a rich and interesting sensory profile, especially an olfactory profile of the composition. In this regard, aroma chemicals which can impart a woody note, fruity note, herbal note, green note, rooty note, citrus note, petitgrain note, floral note, damascone note, tobacco note, ashtray note, gourmand note, sweet note, vetiver note, ionone note, ambra note, fresh note, natural note, Fir Needle note, camphoraceous note, or any combination of two or more of these impressions are of major interest. In addition, the substantivity as well as the tenacity are of special interest in order to obtain a long-lasting odiferous impression in the composition as well as to the surface with which the composition is treated.

However, since even small changes in chemical structure bring about massive changes in the sensory properties such as odor and/or flavor, the targeted search for substances with certain and distinct sensory properties such as a certain odor is extremely difficult. The search for new aroma chemicals is therefore in most cases difficult and laborious without knowing whether a substance with the desired odor and/or flavor will even actually be found.

It is an object of the presently claimed invention to provide substances which can be used as an aroma chemical either alone or as mixtures in compositions, in particular odor-intensive substances having a pleasant odor are sought. Furthermore, they should be combinable with other aroma chemicals, allowing the creation of novel advantageous sensory profiles and can be used in compositions.

It is an object of the presently claimed invention to provide a new aroma chemical which has pleasant olfactory impression; preferably a combination of two or more of the impression which are selected from the group consisting of woody note, fruity note, herbal note, green note, rooty note, citrus note, petitgrain note, floral note, damascone note, tobacco note, ashtray note, gourmand note, sweet note, vetiver note, ionone note, ambra note, natural note, fir needle note, camphoraceous note, or fresh note.

A further object of the present invention is that the aroma chemicals should be obtainable from readily available starting materials, allowing their fast and economic manufacturing.

### SUMMARY OF THE INVENTION

These objects were achieved by the provision of inventive compounds and/or mixtures of the present invention. It was surprisingly found that the compounds of the present invention had a pleasant olfactory impression; preferably a combination of two or more of the impressions which are selected from the group consisting of woody note, fruity note, herbal note, green note, rooty note, citrus note, petitgrain note, floral note, damascone note, tobacco note, ashtray note, gourmand note, sweet note, vetiver note, ionone note, ambra note, natural note, fir needle note, camphoraceous note, or fresh note.

A first aspect of the presently claimed invention relates to the use of a compound of formula (I)

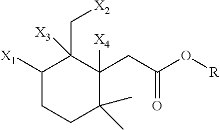

compound of formula (I)
or stereoisomers thereof,
where,
X₁ and X₃ together form a double bond between the carbon atoms to which they are bound, with the proviso that X₂ and X₄ are hydrogen; or
X₃ and X₄ together form a double bond between the carbon atoms to which they are bound, with the proviso that X₁ and X₂ are hydrogen; or
X₂ and X₃ together form a double bond between the carbon atoms to which they are bound, with the proviso that X₂ and X₄ are hydrogen, or
X₄ is OH, X₁, X₂ and X₃ are all hydrogen,
and where R is selected from C₁-C₅ linear or branched alkyl and C₃-C₅ linear or branched alkenyl
   to impart an aroma impression to a composition.

A second aspect of the presently claimed invention relates to the use of compound of formula (I) which comprises,
at least one compound of formula (Ia)

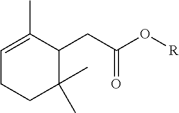

compound of formula (Ia)
or its stereoisomers or mix of its stereoisomers,
or at least one compound of formula (Ib)

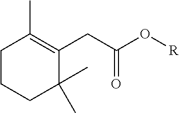

compound of formula (Ib)
or at least one compound of formula (Ic)

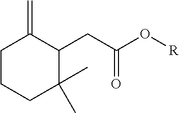

compound of formula (Ic)
or its stereoisomer or mix of its stereoisomers,
or at least one compound of formula (Id)

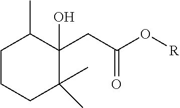

compound of formula (Id)
or its stereoisomer or mix of its stereoisomers,
or mixtures thereof;
where
R is selected from C₁-C₅ linear or branched alkyl and C₃-C₅ linear or branched alkenyl, to impart an aroma impression to a composition.

A third aspect of the presently claimed invention relates to the use of a mixture of at least one compound of formula (Ia)

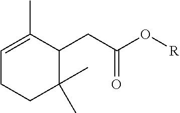

compound of formula (Ia)
or its stereoisomers or mix of its stereoisomers,
and at least one compound of formula (Ib),

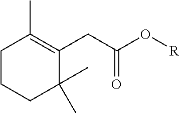

compound of formula (Ib)
R is selected from C₁-C₅ linear or branched alkyl and C₃-C₅ linear or branched alkenyl, to impart an aroma impression to a composition.

A fourth aspect of the presently claimed invention relates to the use of the mixture of at least one compound of formula (Ia)

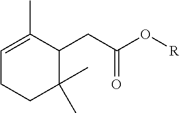

compound of formula (Ia)
or its stereoisomers or mix of its stereoisomers,
at least one compound of formula (Ib),

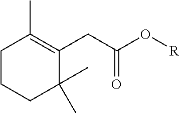

compound of formula (Ib)

and

at least one compound of formula (Ic)

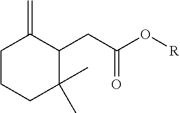

compound of formula (Ic)

or its stereoisomers or mix of its stereoisomers,

where

R is selected from C₁-C₅ linear or branched alkyl and C₃-C₅ linear or branched alkenyl, to impart an aroma impression to a composition.

A further aspect of the presently claimed invention relates to a method of imparting an aroma impression to a composition comprising at least the step of adding at least one compound of formula (I) to a composition.

Another aspect of the presently claimed invention relates to the use of at least one compound of formula (I) for modifying the aroma character composition.

A further aspect of the present invention relates to a mixture comprising at least one compound of formula (Ib),

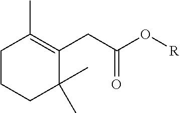

compound of formula (Ib),
and
at least one compound of formula (IV)

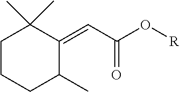

compound of formula (IV)
or its stereoisomers or mix of its stereoisomers,
where,
R is selected from C₁-C₅ linear or branched alkyl and C₃-C₅ linear or branched alkenyl.

A further aspect of the present invention relates to the use of compound of formula (Id)

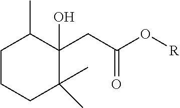

compound of formula (Id)
or its stereoisomers or mix of its stereoisomers,

where

R is selected from C₁-C₅ linear or branched alkyl and C₃-C₅ linear or branched alkenyl, to impart an aroma impression to a composition.

A further aspect of the presently claimed invention relates to a method of boosting the aroma of a composition. Said method comprises the step of mixing at least one compound of formula (I) with other ingredients such as, e.g., at least one other aroma chemical different from the compound of formula (I) and/or at least one non-aroma chemical carrier so as to obtain the aroma chemical composition.

Yet another aspect of the presently claimed invention relates to a method of modifying the aroma of a chemical composition. Said method comprises the step of incorporating at least one compound of the presently claimed invention into an aroma chemical composition to obtain an aroma-modified aroma chemical composition.

Furthermore, the compound of formula (I) can be produced in good yields and purities by a simple synthesis starting from readily available starting materials. Thus, the compounds of the presently claimed invention can be produced in large scales and in a simple and cost-efficient manner.

### DETAILED DESCRIPTION

The following detailed description is exemplary in nature and is not intended to limit the presently claimed invention or the application and uses of the presently claimed invention. Furthermore, there is no intention to be bound by any theory presented in the preceding technical field, background, summary, or the following detailed description.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

Furthermore, the terms “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the subject matter described herein are capable of operation in other sequences than described or illustrated herein. In case the terms “(A)”, “(B)” and “(C)” or AA), BB) and CC) or “(a)”, “(b)”, “(c)”, “(d)”, “(i)”, “(ii)” etc. relate to steps of a method or use or assay there is no time or time interval coherence between the steps, that is, the steps may be carried out simultaneously or there may be time intervals of seconds, minutes, hours, days, weeks, months or even years between such steps, unless otherwise indicated in the application as set forth herein above or below.

In the following passages, different aspects of the subject matter are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” or “preferred embodiment” means that a particular feature, structure or characteristic described in connection with the embodiment is included in at least one embodiment of the presently claimed invention. Thus, appearances of the phrases “in one embodiment” or “In a preferred embodiment” or “in a preferred embodiment” in various places throughout this specification are not necessarily all referring to the same embodiment but may refer. Furthermore, the features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the subject matter, and form different embodiments, as would be understood by those in the art. For example, in the appended claims, any of the claimed embodiments are used in any combination.

Furthermore, the ranges defined throughout the specification include the end values as well, i.e. a range of 1 to 10 implies that both 1 and 10 are included in the range. For the avoidance of doubt, the applicant shall be entitled to any equivalents according to applicable law.

In the context of the present invention, the term “aroma” refers to a sensory property and comprises an odor and/or a flavor.

The term “aroma chemical” denotes a substance which is used to obtain a sensory or organoleptic (used interchangeably herein) impression and comprises its use to obtain an olfactory and/or a flavor impression. The term “olfactory impression” or “note” (used interchangeably here) denotes an odor impression without any positive or negative judgement, while the term “scent impression” or “fragrance impression” or “aroma impression” (used interchangeably herein) as used herein is connected to an odor impression which is generally felt as pleasant. Thus a “fragrance” or “scent” denotes an aroma chemical, which predominately induces a pleasant odor impression. A flavor denotes an aroma chemical, which induces a taste impression.

The term “aroma composition”, as used herein, refers to a composition which induces an aroma. The term aroma composition comprises “odor composition” and/or “flavor composition”. An odor composition being a composition, which predominately induces an odor impression, a flavor composition being a composition, which predominantly induces a taste impression.

The term “aroma profile” denotes the overall aroma impression of an aroma chemical and is composed of the individual aroma impressions of an aroma chemical.

The term odor composition comprises “fragrance composition” or “scent composition” (used interchangeably herein), which predominately induce an odor impression which is generally felt as pleasant.

The general expressions “advantageous sensory properties” or “advantageous organoleptic properties” describe the niceness and conciseness of an organoleptic impression conveyed by an aroma chemical. “Niceness” and “conciseness” are terms which are familiar to the person skilled in the art, such as a perfumer. Niceness generally refers to a spontaneously brought about, positively perceived, pleasant sensory impression. However, “nice” does not have to be synonymous with “sweet”. “Nice” can also be the odor of musk or sandalwood. “Conciseness” generally refers to a spontaneously brought about sensory impression which—for the same test panel-brings about a reproducibly identical reminder of something specific. For example, a substance can have an odor which is spontaneously reminiscent of that of an “apple”: the odor would then be concisely of “apples”. If this apple odor were very pleasant because the odor is reminiscent, for example, of a sweet, fully ripe apple, the odor would be termed “nice”. However, the odor of a typically tart apple can also be concise. If both reactions arise upon smelling the substance, in the example thus a nice and concise apple odor, then this substance has particularly advantageous sensory properties.

The expressions “combination of”, “in combination with” or “combined with” when used herein referring to the compositions, methods or the use of two compounds, take account of the fact that the two compounds do not need to be used in the form of a physical mixture of said compounds but can be used (e.g., added) separately. Where the compounds are used separately, they can be used (e.g. added) sequentially (i.e. one after the other) in any order, or concurrently (i.e. basically at the same time).

The term “boosting”, or “boost” is used herein to describe the effect of enhancing and/or modifying the aroma of an aroma chemical or of a composition. The term “enhancing” comprises an improvement of the niceness and/or conciseness of an aroma and/or an improvement of the intensity. The term “modifying” comprises the change of an aroma profile. The terms “niceness” and “conciseness” are familiar to the person skilled in the art, such as a perfumer and have the respective meaning.

The intensity can be determined via a threshold value determination. A threshold value of an odor is the concentration of a substance in the relevant gas space at which an odor impression can just still be perceived by a representative test panel, although it no longer has to be defined.

Booster effects are particularly desired in fragrance composition when top-note-characterized applications are required, in which the odor is to be conveyed particularly quickly and intensively, for example in deodorants, air fresheners or in the taste sector in chewing gums.

The terms “the invention relates to” and “the invention is directed to” are used synonymously throughout the invention.

The term “tenacity” describes the evaporation behaviour over time of an aroma chemical. The tenacity can for example be determined by applying the aroma chemical to a test strip, and by subsequent olfactory evaluation of the odor impression of the test strip. For aroma chemicals with high tenacity the time span after which the panel can still identify an aroma impression is long.

The term “substantivity” describes the interaction of an aroma chemical with a surface, such as for example the skin or a textile, especially after subsequent treatment of the surface, such as for example washing. The substantivity can for example be determined by washing a textile with a textile detergent composition comprising the aroma chemical and subsequent olfactory evaluation of the textile directly after washing (wet textile) as well as evaluation of the dry textile after prolonged storage.

The term “stability” describes the behaviour of an aroma chemical upon contact with oxygen, light and/or other substances. An aroma chemical with high stability maintains its aroma profile over a long period in time, preferably in a large variety of compositions and under various storage conditions.

In order to impart a long-lasting aroma impression to a composition or to a surface treated with a composition, the tenacity, the substantivity as well as the stability of the aroma chemical in the compositions should preferably be high.

The term C₁-C₅-alkyl denotes a linear or branched alkyl radical comprising 1 to 5 carbon atoms, such as methyl, ethyl, propyl, 1-methylethyl (isopropyl), butyl,1-methylpropyl, 2 methyl propyl, 1,1-dimethylethyl, pentyl, 1-methylbutyl, 2-methylbutyl, 3-methylbutyl, 2,2-dimethylpropyl, 1-ethylpropyl.

The term “C₃-C₅-alkenyl” refers to a straight-chain or branched unsaturated hydrocarbon radical having 3 to 5 carbon atoms and a double bond in any position.

Examples are “C₃-C₅-alkenyl” groups, such as 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl.

The terms “cyclohomogeranate” and “ester of cyclohomogeranic acid” are used interchangeably in the present specification.

As used herein the terms “compound (I)” or “compounds (I)” refer to the compound(s) of formula (1) including, all the stereoisomeric forms (stereoisomers) thereof in all ratios and/or the salts thereof.

The term “mixture” used herein denotes a mixture of at least one compound of formula (Ia) or its stereoisomers or mix of its stereoisomers and/or at least one compound of formula (Ib) and/or compound of formula (Ic).

The compound of formula (Ia) and (1c) refers to the compound in its racemic form, or one of its enantiomerically pure forms (Ror S), or as a mixture of the two possible enantiomers in any ratio, where the ratio of the enantiomers is in the range of 0.01:99.99 to 99.99 to0.01.

The term “stereoisomer” is a general term as described by IUPAC that is used for all isomers of individual compounds that differ only in the arrangement of their atoms in space, not in the connectivity of the atoms. Thus, the term stereoisomer includes mirror image isomers (enantiomers), geometric (cis/trans or E/Z) isomers, and diastereoisomers. For precise definitions of the terms, see the IUPAC definition or G. Helmchen: “Vocabulary and Nomenclature of Organic Stereochemistry” in Houben-Weyl E21a, Stereoselective Synthesis. Helmchen, R. W. Hoffmann, J. Mulzer, E. Schaumann (Hrsg.), 1995, 1-74. The possible isomers can be present as mixtures (i.e. racemates, cis/trans-mixtures or mixtures of diasteroisomers).

### Use

One embodiment the presently claimed invention is direct to the use of a compound of formula (1)

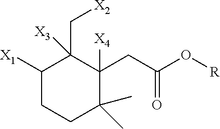

compound of formula (I)
or stereoisomers thereof
where,
X₁ and X₃ together form a double bond between the carbon atoms to which they are bound, with the proviso that X₂ and X₄ are hydrogen; or
X₃ and X₄ together form a double bond between the carbon atoms to which they are bound, with the proviso that X₁ and X₂ are hydrogen; or
X₂ and X₃ together form a double bond between the carbon atoms to which they are bound, with the proviso that X₁ and X₄ are hydrogen; or
X₄ is OH, X₁, X₂ and X₃ are all hydrogen,
where

R is selected from C₁-C₅ linear or branched alkyl and C₃-C₅ linear or branched alkenyl, to impart an aroma impression to a composition.

In an embodiment the presently claimed invention relates to the use of compound of formula (I) which comprises,
at least one compound of formula (Ia)

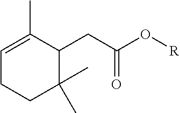

Compound of formula (Ia)
or its stereoisomers or mix of its stereoisomers
or at least one Compound of formula (Ib),

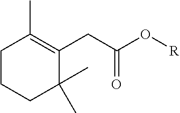

Compound of formula (Ib)
or at least one compound of formula (Ic),

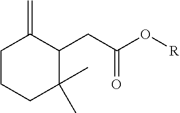

compound of formula (Ic)
or its stereoisomers or mix of its stereoisomers,
or at least one compound of formula (Id)

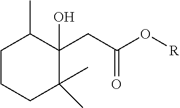

compound of formula (Id)
or its stereoisomers or mix of its stereoisomers,

or mixtures thereof;

where,

R is selected from C₁-C₅ linear or branched alkyl and C₃-C₅ linear or branched alkenyl, to impart an aroma impression to a composition.

In another embodiment of the presently claimed invention relates to the use of a mixture of at least one compound of formula (Ia)

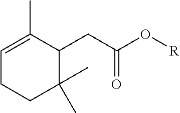

Compound of formula (Ia)
or its stereoisomers or mix of its stereoisomers; and
at least one Compound of formula (Ib),

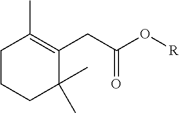

Compound of formula (Ib)
where
R is selected from C₁-C₅ linear or branched alkyl and C₃-C₅ linear or branched alkenyl, to impart an aroma impression to a composition.

A further embodiment of the presently claimed invention relates to the use of the mixture of at least one compound of formula (Ia)

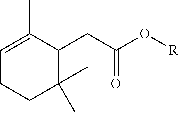

Compound of formula (Ia)
or its stereoisomers or mix of its stereoisomers,
at least one compound of formula (Ib),

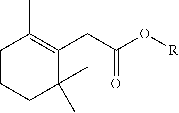

Compound of formula (Ib)
and, at least one compound of formula (Ic)

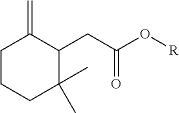

compound of formula (Ic),
or its stereoisomers or mix of its stereoisomers,
where
R is selected from C₁-C₅ linear or branched alkyl and C₃-C₅ linear or branched alkenyl, to impart an aroma impression to a composition.

In an embodiment R is selected from methyl, ethyl, propyl, butyl, isobutyl, isopropyl, 1-propenyl, or 2-propenyl.

Preferably R is methyl, ethyl or 1-propenyl.

In an embodiment compound of formula (Ia) and compound of formula (Ib) can be used alone or in a mixture in any proportion.

In another embodiment, the mixture of compound of formula (Ia) and compound of formula (Ib) is a mixture wherein the ratio of the compounds are in the range of 0.1:99.9 to 99.9:0.1.

In another embodiment, the mixture of compound of formula (Ia) and compound of formula (Ib) is a mixture wherein the ratio of the compounds are in the range of 0.1:99.9 to 99.9:0.1 or any any integer from 0.1 to 99.9.

In another embodiment, the mixture of compound of formula (Ia) and compound of formula (Ib) is a mixture wherein the ratio of the compounds are in the range of 1:99 to 99:1. Preferably in the range of 20:80 to 80:20, or in the range of 30:70 to 70:30, or in the range of 40:60 to 60:40.

In another embodiment, the mixture of compound of formula (Ia) and compound of formula (Ib) is a mixture wherein the ratio of the compounds is in the ratio of 50:50.

A person skilled in the art can adjust the ratios of compound of formula (Ia) and compound of formula (Ib) to arrive at a particular ratio within the range of 0.1:99.9 to 99.9:0.1 to impart a specific aroma impression or several aroma impressions to a composition.

In an embodiment, the compound of formula (Ia) refers to the compound in its racemic form, or one of its enantiomerically pure forms (R or S), or as a mixture of the two possible enantiomers in any ratio, where the ratio of the enantiomers in in the range of 0.01:99.99 to 99.99 to 0.01.

In another embodiment, the mixture has a ratio in the range of 90:10 to 99.9:0.1, wherein the ratio represents the combined amount of compound of formula (Ia) and compound of formula (Ib) to the compound of formula (Ic).

In an embodiment, the mixture has a ratio of,

[Compund of formula (Ia)+comppund of formula (Ib)]: [compound of formula (Ic)] in the range of 90:10 to 99.9:0.1, preferbly in the range of 93:7 to 99:1, more preferbly in the range of 95:5 to 99.9:0.1.

A preferred embodiment of the presently claimed invention is direct to the use of at least one compound of formula (I) as aroma chemical to impart a of woody note, fruity note, herbal note, green note, rooty note, citrus note, petitgrain note, floral note, damascone note, tobacco note, ashtray note, gourmand note, sweet note, vetiver note, ionone note, ambra note, fresh note, natural note, Fir Needle note, camphoraceous note, or any combination of two or more of these to a composition.

In a further embodiment the presently claimed invention is directed to the use of compound of formula (I) to boost the aroma of a composition.

In an embodiment of the presently claimed invention, at least one compound of formula (I) is used as a fragrance or fragrance composition.

In particular, at least one compound of formula (I) is used to impart a note that is selected from the group consisting woody note, fruity note, herbal note, green note, rooty note, citrus note, petitgrain note, floral note, damascone note, tobacco note, ashtray note, gourmand note, sweet note, vetiver note, ionone note, ambra note, fresh note, natural note, Fir Needle note, camphoraceous note, or a combination of two or more notes to a composition.

In an embodiment, the present invention relates to a mixture comprising at least one compound of formula (Ib),

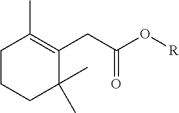

compound of formula (Ib),
or its stereoisomers or mix of its stereoisomers,
and
at least one compound of formula (IV)

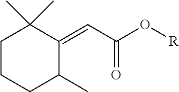

compound of formula (IV)
or its stereoisomers or mix of its stereoisomers where,
R is selected from C₁-C₅ linear or branched alkyl and C₃-C₅ linear or branched alkenyl.

In another embodiment, the present invention relates to the use of a mixture
comprising at least one compound of formula (Ib).

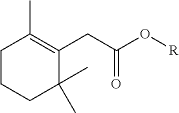

Compound of formula (Ib),
and
at least one compound of formula (IV)

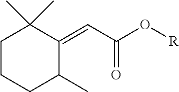

Compound of formula (IV)
or its stereoisomers or mix of its stereoisomers where,
R is selected from C₁-C₅ linear or branched alkyl and C₃-C₅ linear or branched alkenyl.
to impart an aroma impression to a composition.

In an embodiment, R is methyl.

In an embodiment, the mixture of compound of formula (Ib) and compound of formula (IV) wherein the weight ratio of the of compound of formula (Ib) and compound of formula (IV) is in the range 90:10 to 99:1.

In an embodiment, the mixture of compound of formula (Ib) and compound of formula (IV) has an aroma impression selected from leather or plastic or a combination thereof.

In an embodiment, the present invention relates to the use of compound of formula (Id)

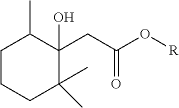

compound of formula (Id)
or its stereoisomers,

where

R is selected from C₁-C₅ linear or branched alkyl and C₃-C₅ linear or branched alkenyl, to impart an aroma impression to a composition.

In an embodiment, the present invention relates to the use of compound of formula (Id)

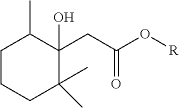

compound of formula (Id)
or its stereoisomers,

where R is methyl.

Preferably compounds of formula (I) are,

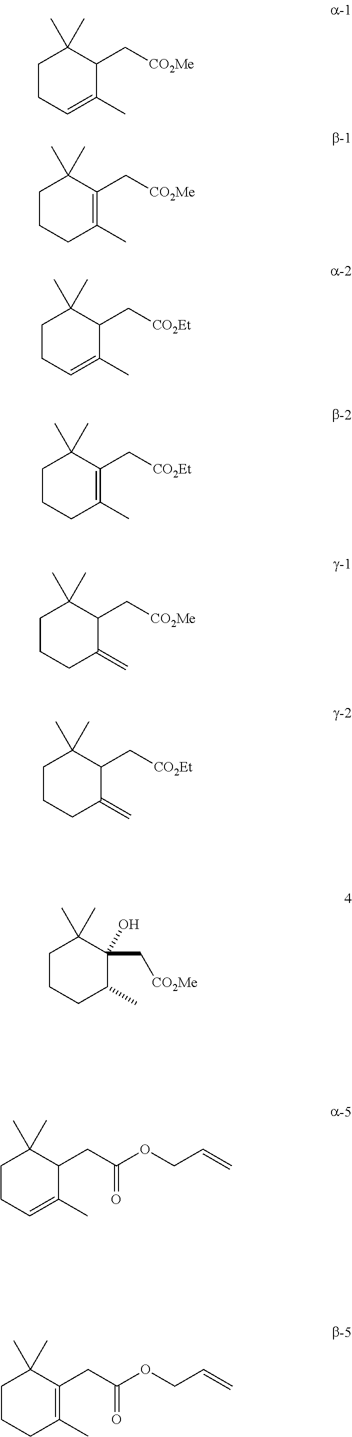

Suitable compositions as described above are for example compositions used in personal care, in home care, in industrial applications as well as compositions used in other applications, such as pharmaceutical compositions or crop protection compositions.

In an embodiment, the compound of formula (I) includes at least one compound of formula (Ia), at least one compound of formula (Ib) and at least one compound of formula (Ic) in a ratio wherein each of the compounds Ia, Ib and 1c could vary between 0.1 to 99.9.

Preferably, the compound of formula (I) according to the presently claimed invention are used in a composition selected from the group consisting of perfume compositions, body care compositions (including cosmetic compositions and products for oral and dental hygiene), hygiene articles, cleaning compositions (including dishwashing compositions), textile detergent compositions, compositions for scent dispensers, foods, food supplements, pharmaceutical compositions and crop protection compositions.

The compositions have been described in the below paragraphs.

### Composition

In an embodiment, the presently claimed invention relates to a composition comprising at least one compound of formula (I) and
(i) at least one aroma chemical (X) different from the compound of formula (I), or
(ii) at least one non-aroma chemical carrier, or
(iii) both of (i) and (ii).

Preferably in this embodiment, the composition comprises compounds of formula (I), in a total amount in the range of >0.01 wt. % to ≤70.0 wt. %, based on the total weight of the composition.

Preferably, the composition is an aroma composition, more preferable a fragrance composition.

Aroma chemical different from the compounds of formula (I).

In one embodiment, the mixture comprises at least one aroma chemical which is different from the compounds of formula (I). Aroma chemicals which are different from the compounds of formula (I) are also referred to as aroma chemical (X).

By virtue of their physical properties, the compounds of formula (I) are well combinable with aroma chemicals which are different from the compounds of formula (I) and other customary ingredients in aroma compositions, in particular fragrance compositions. This allows, e.g., the creation of aroma compositions (preferably fragrance compositions) which have novel advantageous sensory profiles. Especially, as already explained above, the compounds can provide a booster effect for other aroma chemicals (such as fragrances).

The aroma chemical (X) is preferably selected from the group consisting of:
geranyl acetate, alpha-hexylcinnamaldehyde, 2-phenoxyethyl isobutyrate, dihydromyrcenol, methyl dihydrojasmonate, 4,6,6,7,8,8 hexamethyl-1,3,4,6,7,8-hexa-hydrocyclopenta[g] benzo-pyran, tetrahydrolinalool, ethyllinalool, benzyl salicylate, 2 methyl-3-(4-tert-butylphenyl) propanal, cinnamyl alcohol, 4,7 methano-3a,4,5,6,7,7a-hexahydro-5 indenyl acetate and/or 4,7 methano-3a,4,5,6,7,7a-hexahydro-6-indenyl acetate, citronellol, citronellyl acetate, tetrahydrogeraniol, vanillin, linalyl acetate, styrolyl acetate, octahydro-2,3,8,8-tetramethyl-2-acetonaphthone and/or 2 acetyl-1,2,3,4,6,7,8-octahydro-2,3,8,8-tetramethylnaphthalene, hexyl salicylate, 4 tert-butylcyclohexyl acetate, 2-tert-butylcyclohexyl acetate, alpha-ionone, alpha-methylionone, alpha-iso-methylionone, coumarin, terpinyl acetate, 2 phenylethyl alcohol, 4-(4-hydroxy-4-methylpentyl)-3-cyclohexene-carboxaldehyde, alpha-amylcinnamaldehyde, ethylene brassylate, (E) and/or (Z)-3-methylcyclopentadec-5 enone, 15-pentadec-11-enolide and/or 15-pentadec-12-enolide, 15-cyclopentadecanolide, 1-(5,6,7,8-tetrahydro-3,5,5,6,8,8-hexamethyl-2-naphthalenyl) ethanone, 2-isobutyl-4-methyltetrahydro-2H pyran-4-ol, 2-ethyl-4-(2,2,3-trimethyl-3-cyclopenten-1-yl)-2-buten-1-ol, cis-3-hexenyl acetate, trans-3-hexenyl acetate, trans-2/cis-6-nonadienol, 2,4-dimethyl-3-cyclohexenecarboxaldehyde, 2,4,4,7-tetramethyloct-6-en-3-one, 2,6-dimethyl-5-hepten-1-al, borneol, 3 (3 isopropylphenyl) butanal, 2-methyl-3-(3,4-methylenedioxyphenyl) propanal, 3-(4-ethylphenyl)-2,2-dimethylpropanal, 7-methyl-2H 1,5-benzodioxepin-3 (4H)-one, 3,3,5-trimethylcyclohexyl acetate, 2,5,5 trimethyl-1,2,3,4,4a,5,6,7-octahydronaphthalen-2-ol, 3-(4-tert-butylphenyl)-propanal, ethyl 2-methylpentanoate, ethoxymethoxycyclododecane, 2,4-dimethyl-4,4a,5,9b-tetrahydroindeno[1,2-d][1,3]dioxine, (2-tert-butylcyclohexyl) acetate and 3-[5,5,6-trimethylbicyclo[2.2.1]hept-2-yl]cyclohexan-1-ol.

In a preferred embodiment, the at least one aroma chemical (i) is selected from the group consisting of methyl benzoate, benzyl acetate, geranyl acetate, 2-isobutyl-4-methyltetrahydro-2H-pyran-4-ol, linalool, 2-isobutyl-4-methyltetrahydro-2H-pyran-4-ol and methyl benzoate.

In another preferred embodiment, the at least one aroma chemical (i) is selected from the group consisting of ethylvanillin, vanillin, 2,5-dimethyl-4-hydroxy-2H-furan-3-one (furaneol) and 3-hydroxy-2-methyl-4H-pyran-4-one (maltol).

Further aroma chemicals with which the compound of formula can be combined to give a composition according to the presently claimed invention can be found, e.g., in S. Arctander, Perfume and Flavor Chemicals, Vol. I and II, Montclair, N. J., 1969, self-published or H. Surburg and J. Panten, Common Fragrance and Flavor Materials, 4th Ed., Wiley-VCH, Weinheim 2016. Specifically, mention may be made of: extracts from natural raw materials such as essential oils, concretes, absolutes, resins, resinoids, balsams, tinctures such as e.g. ambergris tincture; amyris oil; angelica seed oil; angelica root oil; aniseed oil; valerian oil; basil oil; tree moss absolute; bay oil; mugwort oil; benzoin resin; bergamot oil; beeswax absolute; birch tar oil; bitter almond oil; savory oil; buchu leaf oil; cabreuva oil; cade oil; calmus oil; camphor oil; cananga oil; cardamom oil; cascarilla oil; cassia oil; cassia absolute; castoreum absolute; cedar leaf oil; cedar wood oil; cistus oil; citronella oil; lemon oil; copaiba balsam; copaiba balsam oil; coriander oil; costus root oil; cumin oil; cypress oil; davana oil; dill weed oil; dill seed oil; Eau de brouts absolute; oak moss absolute; elemi oil; tarragon oil; eucalyptus citriodora oil; eucalyptus oil; fennel oil; pine needle oil; galbanum oil; galbanum resin; geranium oil; grapefruit oil; guaiacwood oil; gurjun balsam; gurjun balsam oil; helichrysum absolute; helichrysum oil; ginger oil; iris root absolute; iris root oil; jasmine absolute; calmus oil; camomile oil blue; roman camomile oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemongrass oil; lovage oil; lime oil distilled; lime oil pressed; linalool oil; litsea cubeba oil; laurel leaf oil; mace oil; marjoram oil; mandarin oil; massoia bark oil; mimosa absolute; musk seed oil; musk tincture; clary sage oil; nutmeg oil; myrrh absolute; myrrh oil; myrtle oil; clove leaf oil; clove flower oil; neroli oil; olibanum absolute; olibanum oil; opopanax oil; orange blossom absolute; orange oil; origanum oil; palmarosa oil; patchouli oil; perilla oil; peru balsam oil; parsley leaf oil; parsley seed oil; petitgrain oil; peppermint oil; pepper oil; pimento oil; pine oil; pennyroyal oil; rose absolute; rose wood oil; rose oil; rosemary oil; Dalmatian sage oil; Spanish sage oil; sandalwood oil; celery seed oil; spike-lavender oil; star anise oil; styrax oil; tagetes oil; fir needle oil; tea tree oil; turpentine oil; thyme oil; tolubalsam; tonka absolute; tuberose absolute; vanilla extract; violet leaf absolute; verbena oil; vetiver oil; juniper berry oil; wine lees oil; wormwood oil; winter green oil; hyssop oil; civet absolute; cinnamon leaf oil; cinnamon bark oil, and fractions thereof, or ingredients isolated therefrom;

individual fragrances from the group of hydrocarbons, such as e.g. 3 carene; alpha-pinene; beta-pinene; alpha-terpinene; gamma-terpinene; p-cymene; bisabolene; camphene; caryophyllene;

cedrene; farnesene; limonene; longifolene; myrcene; ocimene; valencene; (E,Z)-1,3,5-undecatriene; styrene; diphenylmethane;

the aliphatic alcohols such as e.g. hexanol; octanol; 3-octanol; 2,6-dimethylheptanol; 2-methyl-2-heptanol; 2-methyl-2-octanol; (E)-2-hexenol; (E)- and (Z)-3-hexenol; 1 octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;

the aliphatic aldehydes and acetals thereof such as e.g. hexanal; heptanal; octanal; nonanal;

decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10 trimethyl-5,9-undecadienal; heptanal diethylacetal; 1,1-dimethoxy-2,2,5 trimethyl-4-hexene; citronellyloxyacetaldehyde; (E/Z)-1-(1-methoxypropoxy)-hex-3-ene; the aliphatic ketones and oximes thereof such as e.g. 2-heptanone; 2-octanone; 3-octanone; 2-nonanone; 5-methyl-3-heptanone; 5-methyl-3 heptanone oxime; 2,4,4,7-tetramethyl-6-octen-3-one; 6-methyl-5-hepten-2-one;

the aliphatic sulfur-containing compounds such as e.g. 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;

the aliphatic nitriles such as e.g. 2-nonenenitrile; 2-undecenenitrile; 2 tridecenenitrile; 3,12-tridecadienenitrile; 3,7-dimethyl-2,6-octadienenitrile; 3,7-dimethyl-6 octenenitrile;

the esters of aliphatic carboxylic acids such as e.g. (E)- and (Z)-3-hexenyl formate; ethyl acetoacetate; isoamyl acetate; hexyl acetate; 3,5,5-trimethylhexyl acetate; 3 methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octinate; methyl 2-noninate; allyl 2-isoamyloxy acetate; methyl-3,7-dimethyl-2,6-octadienoate; 4-methyl-2-pentyl crotonate;

the acyclic terpene alcohols such as e.g. geraniol; nerol; linalool; lavandulol; nerolidol; farnesol;

tetrahydrolinalool; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2 ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates and 3-methyl-2 butenoates thereof;

the acyclic terpene aldehydes and ketones such as e.g. geranial; neral; citronellal; 7 hydroxy-3,7-dimethyloctanal; 7 methoxy-3,7-dimethyloctanal; 2,6,10-trimethyl-9 undecenal; geranyl acetone; as well as the dimethyl and diethylacetals of geranial, neral, 7-hydroxy-3,7-dimethyloctanal; the cyclic terpene alcohols such as e.g. menthol; isopulegol; alpha-terpineol; terpinene-4-ol;

menthan-8-ol; menthan-1-ol; menthan-7-ol; borneol; isoborneol; linalool oxide; nopol; cedrol;

ambrinol; vetiverol; guajol; and the formates, acetates, propionates, isobutyrates, butyrates, isovalerates, pentanoates, hexanoates, crotonates, tiglinates and 3-methyl-2-butenoates thereof;

the cyclic terpene aldehydes and ketones such as e.g. menthone; isomenthone; 8 mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methano-naphthalene-8 (5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedar wood oil (methyl cedryl ketone);

the cyclic alcohols such as e.g. 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;

the cycloaliphatic alcohols such as e.g. alpha-3,3-trimethylcyclohexylmethanol; 1 (4-isopropylcyclohexyl) ethanol; 2-methyl-4-(2,2,3-trimethyl-3-cyclopent-1-yl) butanol; 2-methyl-4-(2,2,3 trimethyl-3-cyclopent-1-yl)-2-buten-1-ol; 2-ethyl-4-(2,2,3-trimethyl-3 cyclopent-1-yl)-2-buten-1-ol; 3-methyl-5-(2,2,3 trimethyl-3-cyclopent-1-yl) pentan-2 ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl) pentan-3-ol; 1-(2,2,6-trimethylcyclohexyl) hexan-3-ol;

the cyclic and cycloaliphatic ethers such as e.g. cineol; cedryl methyl ether; cyclododecyl methyl ether; 1,1-dimethoxycyclododecane; (ethoxymethoxy) cyclo-dodecane; alpha-cedrene epoxide; 3a, 6,6,9a-tetramethyldodecahydronaphtho[2,1-b]furan; 3a-ethyl-6,6,9a-trimethyldodecahydro-naphtho[2,1-b]furan; 1,5,9-trimethyl-13-oxabicyclo-[10.1.0] trideca-4,8-diene; rose oxide; 2-(2,4-dimethyl-3-cyclohexen-1-yl)-5-methyl-5-(1-methylpropyl)-1,3-dioxane;

the cyclic and macrocyclic ketones such as e.g. 4-tert-butylcyclohexanone; 2,2,5 trimethyl-5-pentylcyclopentanone; 2-heptylcyclopentanone; 2-pentylcyclo-pentanone; 2-hydroxy-3-methyl-2-cyclopenten-1-one; 3-methyl-cis-2-penten-1-yl-2 cyclopenten-1-one; 3-methyl-2-pentyl-2-cyclopenten-1-one; 3-methyl-4-cyclopenta-decenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4 (5H)-indanone; 8-cyclo-hexadecen-1-one; 7-cyclohexadecen-1-one; (7/8)-cyclohexadecen-1-one; 9 cyclo-heptadecen-1-one; cyclopentadecanone; cyclohexadecanone;
the cycloaliphatic aldehydes such as e.g. 2,4-dimethyl-3-cyclohexenecarbaldehyde; 2 methyl-4-(2,2,6-trimethylcyclohexen-1-yl)-2-butenal; 4-(4-hydroxy-4-methylpentyl)-3 cyclohexene carbaldehyde; 4-(4-methyl-3-penten-1-yl)-3-cyclohexenecarbaldehyde;

the cycloaliphatic ketones such as e.g. 1-(3,3-dimethylcyclohexyl)-4-penten-1-one; 2,2 dimethyl-1-(2,4-dimethyl-3-cyclohexen-1-yl)-1-propanone; 1-(5,5-dimethyl-1 cyclo-hexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl (2,4-dimethyl-3-cyclohexen-1-yl) ketone;

the esters of cyclic alcohols such as e.g. 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate;

4,7-methano-3a,4,5,6,7,7a-hexahydro-5 or 6-indenyl acetate; 4,7-methano-3a,4,5,6,7,7a-hexahydro-5 or 6 indenyl propionate; 4,7-methano-3a,4,5,6,7,7a-hexahydro-5 or 6-indenyl isobutyrate; 4,7 methanooctahydro-5 or 6-indenyl acetate;

the esters of cycloaliphatic alcohols such as e.g. 1-cyclohexylethyl crotonate;

the esters of cycloaliphatic carboxylic acids such as e.g. allyl 3-cyclohexylpropionate; allyl cyclohexyloxyacetate; cis and trans-methyl dihydrojasmonate; cis and trans-methyl jasmonate;

methyl 2-hexyl-3-oxocyclopentanecarboxylate; ethyl 2-ethyl-6,6 dimethyl-2-cyclohexenecarboxylate; ethyl 2,3,6,6-tetramethyl-2 cyclohexene-carboxylate; ethyl 2-methyl-1,3-dioxolane-2-acetate;

the araliphatic alcohols such as e.g. benzyl alcohol; 1-phenylethyl alcohol, 2 phenylethyl alcohol, 3-phenylpropanol; 2-phenylpropanol; 2-phenoxyethanol; 2,2-dimethyl-3-phenylpropanol; 2,2-dimethyl-3-(3-methylphenyl) propanol; 1,1-dimethyl-2 phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxy-benzyl alcohol; 1-(4-isopropylphenyl) ethanol;

the esters of araliphatic alcohols and aliphatic carboxylic acids such as e.g. benzyl acetate; benzyl propionate; benzyl isobutyrate; benzyl isovalerate; 2-phenylethyl acetate; 2-phenylethyl propionate; 2-phenylethyl isobutyrate; 2 phenylethyl isovalerate; 1 phenylethyl acetate; alpha-trichloromethylbenzyl acetate; alpha, alpha-dimethylphenylethyl acetate; alpha, alpha-dimethylphenylethyl butyrate; cinnamyl acetate; 2-phenoxyethyl isobutyrate; 4-methoxybenzyl acetate;

the araliphatic ethers such as e.g. 2-phenylethyl methyl ether; 2 phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxine; 4,4a,5,9b-tetrahydro-2,4-dimethylindeno[1,2-d]-m dioxine;

the aromatic and araliphatic aldehydes such as e.g. benzaldehyde; phenylacetaldehyde; 3-phenylpropanal; hydratropaaldehyde; 4-methylbenzaldehyde; 4 methylphenylacetaldehyde; 3-(4-ethylphenyl)-2,2-dimethylpropanal; 2-methyl-3-(4-isopropylphenyl) propanal; 2-methyl-3-(4-tert-butylphenyl) propanal; 2-methyl-3-(4-isobutylphenyl) propanal; 3-(4-tert-butylphenyl) propanal; cinnamaldehyde; alpha-butylcinnamaldehyde; alpha-amylcinnamaldehyde; alpha-hexylcinnamaldehyde; 3 methyl-5-phenylpentanal; 4-methoxybenzaldehyde; 4-hydroxy-3 methoxy-benzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl) propanal; 2-methyl-3-(4-methylenedioxyphenyl) propanal;

the aromatic and araliphatic ketones such as e.g. acetophenone; 4-methylacetophenone; 4-methoxyacetophenone; 4-tert-butyl-2,6-dimethylaceto-phenone; 4-phenyl-2-butanone; 4-(4-hydroxyphenyl)-2-butanone; 1-(2-naphthalenyl)-ethanone; 2-benzofuranylethanone; (3-methyl-2-benzofuranyl) ethanone; benzophenone; 1,1,2,3,3,6-hexamethyl-5-indanyl methyl ketone; 6-tert-butyl-1,1 dimethyl-4 indanyl methyl ketone; 1-[2,3-dihydro-1,1,2,6-tetramethyl-3-(1-methylethyl)-1H-5 indenylJethanone; 5′,6′,7′,8′-Tetrahydro-3′,5′,5′,6′,8′,8′-hexamethyl-2′-acetonaphthone;

the aromatic and aliphatic carboxylic acids and esters thereof such as e.g. benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;

the nitrogen-containing aromatic compounds such as e.g. 2,4,6-trinitro-1,3-dimethyl-5 tert-butylbenzene; 3,5-dinitro-2,6-dimethyl-4-tert-butylacetophenone; cinnamonitrile; 3 methyl-5-phenyl-2-pentenonitrile; 3-methyl-5-phenylpentanonitrile; methyl anthranilate; methyl-N-methylanthranilate; Schiff bases of methyl anthranilate with 7 hydroxy-3,7-dimethyloctanal, 2-methyl-3-(4-tert-butylphenyl) propanal or 2,4 dimethyl-3-cyclohexenecarbaldehyde; 6-isopropylquinoline; 6-isobutylquinoline; 6-sec-butylquinoline; 2-(3-phenylpropyl) pyridine; indole; skatole; 2-methoxy-3 isopropyl-pyrazine; 2-isobutyl-3-methoxypyrazine;

the phenols, phenyl ethers and phenyl esters such as e.g. estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2 ethoxy-5-(1-propenyl) phenol; p-cresyl phenylacetate;

the heterocyclic compounds such as e.g. 2,5-dimethyl-4-hydroxy-2H-furan-3-one; 2 ethyl-4-hydroxy-5-methyl-2H-furan-3-one; 3-hydroxy-2-methyl-4H-pyran-4-one; 2 ethyl-3-hydroxy-4H-pyran-4-one;

the lactones such as e.g. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis and trans-11-pentadecen-1,15-olide; cis and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin; tetrahydroactinidiolide.

The aroma chemical (X) used in the composition are obtained from known commercial sources and procured from Germany.

In a preferred embodiment, the composition comprises at least one compound of formula (I) and at least one aroma chemical (X).

In another preferred embodiment, the composition comprises at least one compound of formula (Ia), at least one compound of formula (Ib) and at least one compound of formula (Ic) and at least one aroma chemical (X).

### Non-Aroma Chemical Carrier:

The non-aroma chemical carrier in the composition of the invention is preferably selected from the group consisting of surfactants, oil components antioxidants, deodorant-active agents and solvents.

Preferably the at least one non-aroma chemical carrier is a compound, a mixture of compounds or other additives, which has/have no or no noteworthy sensory properties. The non-aroma chemical carrier can serve for the dilution and/or the fixing of the compounds of formula (I) and-optionally the at least one aroma chemical (X), as defined above, if comprised in the composition.

The non-aroma chemical carrier in the composition of the invention is preferably selected from the group consisting of surfactants, oil components, solvents or any mixture of two or more of the aforementioned.

### Solvent

In the context of the presently claimed invention, a “solvent” serves for the dilution of the compounds of formula (I) to be used according to the invention without having its own aroma.

The amount of solvent(s) is selected depending on the composition.

Preferably, the solvent is present in the composition in a total amount of 0.01 wt % to 99.0 wt %, more preferably in a total amount of 0.05 wt. % to 95.0 wt. %, yet more preferably in a total amount of 0.1 wt. % to 80.0 wt. %, most preferably 0.1 wt. % to 70.0 wt. %, particularly in a total amount of 0.1 wt. % to 60.0 wt. %, based on the total weight of the composition.

In a preferred embodiment, the composition comprises 0.05 wt. % to 10 wt. %, more preferably 0.1 wt. % to 5 wt. %, yet more preferably 0.2 wt. % to 3 wt. % total solvent(s), based on the total weight of the composition. In yet another preferred embodiment of the invention, the composition comprises 20 wt. % to 70 wt. %, more preferably 25 wt. % to 50 wt. % of total solvent(s), based on the total weight of the composition.

Preferred solvents are selected from the group consisting of ethanol, isopropanol, diethylene glycol monoethyl ether, glycerol, propylene glycol, 1,2 butylene glycol, dipropylene glycol, triethyl citrate, isopropyl myristate and any mixture of two or more of the aforementioned.

In a preferred embodiment, the composition comprises at least one compound of formula (I) and at least one solvent and optionally at least one aroma chemical (X).

In another preferred embodiment, the composition comprises at least one compound of formula (Ia), at least one compound of formula (Ib), and at least one compound of formula (Ic) and at least one solvent and optionally at least one aroma chemical (X).

### Oil Component

Preferably, the total oil components are present in an amount of 0.1 to 80 wt. %, more preferably 0.5 to 70 wt. %, yet more preferably 1 to 60 wt. %, even more preferably 1 to 50 wt. %, particularly 1 to 40 wt. %, more particularly 5 to 25 wt. % and specifically 5 to 15 wt. %, based on the total weight of the composition.

Preferably, the oil components are selected from Guerbet alcohols based on fatty alcohols containing 6 to 18, preferably 8 to 10, carbon atoms and other additional esters, such as myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of C₁₈-C₃₈ alkyl-hydroxycarboxylic acids with linear or branched C₆-C₂₂ fatty alcohols, more especially dioctyl malate, esters of linear and/or branched fatty acids with polyhydric alcohols (for example propylene glycol, dimer dial or trimer triol), triglycerides based on C₆-C₁₀ fatty acids, liquid mono-, di- and triglyceride mixtures based on C₆-C₁₈ fatty acids, esters of C₆-C₂₂ fatty alcohols and/or Guerbet alcohols with aromatic carboxylic acids, more particularly benzoic acid, esters of dicarboxylic acids with polyols containing 2 to 10 carbon atoms and 2 to 6 hydroxyl groups, vegetable oils, branched primary alcohols, substituted cyclohexanes, linear and branched C₆-C₂₂ fatty alcohol carbonates such as, for example, dicaprylyl carbonate (Cetiol® CC), Guerbet carbonates based on fatty alcohols containing 6 to 18, preferably 8 to 10, carbon atoms, esters of benzoic acid with linear and/or branched C₆ to C₂₂ alcohols (for example Finsolv@ TN), linear or branched, symmetrical or nonsymmetrical dialkyl ethers containing 6 to 22 carbon atoms per alkyl group such as, for example, dicaprylyl ether (Cetiol® OE), ring opening products of epoxidized fatty acid esters with polyols and hydrocarbons or mixtures thereof.

In a preferred embodiment, the composition comprises at least one compound of formula (I) and at least one oil component and optionally at least one aroma chemical (X).

In another preferred embodiment, the composition comprises at least one compound of formula (Ia), at least one compound of formula (Ib), and at least one compound of formula (Ic) and at least one oil component and optionally at least one aroma chemical (X).

### Antioxidants

It is to be understood that antioxidants are able to inhibit or prevent the undesired changes in the compositions to be protected caused by oxygen effects and other oxidative processes. The effect of the antioxidants consists in most cases in them acting as free-radical scavengers for the free radicals which arise during autoxidation.

In a preferred embodiment, the antioxidant is selected from the group consisting of
amino acids (for example glycine, alanine, arginine, serine, threonine, histidine, tyrosine, tryptophan) and derivatives thereof,
imidazoles (e.g. urocanic acid) and derivatives thereof,
peptides, such as D,L-carnosine, D-carnosine, L-carnosine (=β-Alanyl-L-histidine) and derivatives thereof,
carotenoids, carotenes (e.g. alpha-carotene, beta-carotene, lycopene, lutein) or derivatives thereof,
chlorogenic acid and derivatives thereof,
lipoic acid and derivatives thereof (for example dihydrolipoic acid),
auro-thioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, gamma-linoleyl, cholesteryl and glyceryl esters thereof) and salts thereof,
dilauryl thiodipropionate, distearyl thiodipropionate, thiodipropionic acid and derivatives thereof (esters, ethers, peptides, lipids, nucleotides, nucleosides and salts),
sulfoximine compounds (for example buthionine sulfoximines, homocysteine sulfoximine, buthionine sulfones, penta-, hexa-, heptathionine sulfoximine),
(metal) chelating agents (e.g. alpha-hydroxy fatty acids, palmitic acid, phytic acid, lactoferrin),
alpha-hydroxy acids (for example citric acid, lactic acid, malic acid),
humic acid, bile acid, bile extracts, bilirubin, biliverdin, boldin (=alkaloid from the plant Peumus boldus, boldo extract,
EDTA, EGTA and derivatives thereof,
unsaturated fatty acids and derivatives thereof (e.g. gamma-linolenic acid, linoleic acid, oleic acid),
folic acid and derivatives thereof,
ubiquinone and ubiquinol and derivatives thereof,
vitamin C and derivatives (for example ascorbyl palmitate, Mg ascorbyl phosphate, ascorbyl acetate),
tocopherols and derivatives (for example vitamin E acetate),
vitamin A and derivatives (for example vitamin A palmitate),
coniferyl benzoate of gum benzoin, rutic acid and derivatives thereof, alpha-glycosylrutin, ferulic acid, furfurylideneglucitol,
butylhydroxytoluene (BHT), butylhydroxyanisole (BHA),
nordihydroguaiacic acid, nordihydroguaiaretic acid, trihydroxybutyrophenone, uric acid and derivatives thereof, mannose and derivatives thereof,
superoxide dismutase,
zinc and derivatives thereof (for example ZnO, ZnSO4),
selenium and derivatives thereof (for example selenomethionine),
stilbenes and derivatives thereof (e.g. stilbene oxide, trans-stilbene oxide) and mixtures of two or more of the aforementioned.

In a preferred embodiment, the anti-oxidant is selected from the group consisting of pentaerythrityl, tetra-di-t-butyl-hydroxyhydrocinnamate, nordihydroguaiaretic acid, ferulic acid, resveratrol, propyl gallate, butylhydroxytoluene (BHT), butylhydroxyanisole (BHA), ascorbyl palmitate, tocopherol and mixtures of two or more of the aforementioned.

Preferably, the compositions according to the presently claimed invention comprise the anti-oxidant in a total amount of 0.001 to 25 wt.-%, preferably 0.005 to 10 wt.-%, more preferably 0.01 to 8 wt.-%, yet more preferably 0.025 to 7 wt.-%, even more preferably 0.05 to 5 wt.-%, based on the total weight of the composition.

In a preferred embodiment, the composition comprises at least one compound of formula (I) and at least one antioxidant and optionally at least one aroma chemical (X).

In another preferred embodiment, the composition comprises at least one compound of formula (Ia), at least one compound of formula (Ib), and at least one compound of formula (Ic), and at least one anti-oxidant and optionally at least one aroma chemical (X).

### Deodorant-Active Agents

Deodorizing compositions (deodorants and antiperspirants) counteract, mask or eliminate body odors. Body odors are formed through the action of skin bacteria on apocrine perspiration which results in the formation of unpleasant-smelling degradation products.

Preferably the deodorant-active agent is selected from the groups consisting of antiperspirants, esterase inhibitors, antibacterial agents and mixtures of two or more of the aforementioned.

Suitable antiperspirants are selected from the group consisting of salts of aluminum, zirconium or zinc. Examples are aluminum chloride, aluminum chlorohydrate, aluminum dichlorohydrate, aluminum sesquichlorohydrate and complex compounds thereof, for example with 1,2-propylene glycol, aluminum hydroxyallantoinate, aluminum chloride tartrate, aluminum zirconium trichlorohydrate, aluminum zirconium tetrachlorohydrate, aluminum zirconium pentachlorohydrate and complex compounds thereof, for example with amino acids, such as glycine. Aluminum chlorohydrate, aluminum zirconium tetrachlorohydrate, aluminum zirconium pentachlorohydrate and complex compounds thereof are preferably used.

Preferably, the anti-perspirant is selected from the group consisting of aluminum chloride, aluminum chlorohydrate, aluminum dichlorohydrate, aluminum sesquichlorohydrate, aluminum hydroxyallantoinate, aluminum chloride tartrate, aluminum zirconium trichlorohydrate, aluminum zirconium tetrachlorohydrate aluminum zirconium pentachlorohydrate and mixtures of two or more of the aforementioned.

Where perspiration is present in the underarm region, extracellular enzymes-esterases, mainly proteases and/or lipases are formed by bacteria and split the esters present in the perspiration, releasing odors in the process. Suitable esterase inhibitors are for example trialkyl citrates, such as trimethyl citrate, tripropyl citrate, triisopropyl citrate, tributyl citrate and, in particular, triethyl citrate. Esterase inhibitors inhibit enzyme activity and thus reduce odor formation. The free acid is probably released by the cleavage of the citric acid ester and reduces the pH value of the skin to such an extent that the enzymes are inactivated by acylation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, zinc glycinate and mixtures of two or more of the aforementioned.

Preferably, the esterase inhibitor is selected from the group consisting of trimethyl citrate, tripropyl citrate, triisopropyl citrate, tributyl citrate triethyl citrate, lanosterol, cholesterol, campesterol, stigmasterol, sitosterol sulfate, sitosterol phosphate, glutaric acid, glutaric acid monoethyl ester, glutaric acid diethyl ester, adipic acid, adipic acid monoethyl ester, adipic acid diethyl ester, malonic acid, malonic acid diethyl ester, citric acid, malic acid, tartaric acid, tartaric acid diethyl ester zinc glycinate and mixtures of two or more of the aforementioned.

Preferably, the compositions according to the presently claimed invention comprise the esterase inhibitor in a total amount in the range of 0.01 to 20 wt.-%, preferably 0.1 to 10 wt.-% and more particularly 0.5 to 5 wt.-%, based on the total weight of the composition.

The term “anti-bacterial agents” as used herein encompasses substances which have bactericidal and/or bacteriostatic properties. Typically these substances act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

Preferably, the antibacterial agent is selected from the group consisting of chitosan, phenoxyethanol, 5-chloro-2-(2,4-dichlorophenoxy)-phenol, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), phenoxyethanol, glycerol monocaprate, glycerol monocaprylate, glycerol monolaurate (GML), diglycerol monocaprate (DMC), salicylic acid-N-alkylamides and mixtures of two or more of the aforementioned.

Preferably, the composition according to the presently claimed invention comprises the antibacterial agent(s) in a total amount in the range of 0.01 to 5 wt. % and preferably 0.1 to 2 wt.-%, based on the total weight of the composition.

In a preferred embodiment, the composition comprises at least one compound of formula (I) and at least one deodorant active agent and optionally at least one aroma chemical (X).

In another preferred embodiment, the composition comprises at least one compound of formula (Ia), at least one compound of formula (Ib), and at least one compound of formula (Ic) and at least one deodorant active agent and optionally at least one aroma chemical (X).

### Surfactants

Preferably, the surfactant is selected from the group consisting of anionic, non-ionic, cationic, amphoteric, zwitterionic surfactant and a mixture of two or more of the aforementioned. More preferably, the surfactant is an anionic surfactant.

Preferably, the compositions according to the invention contain the surfactant(s), in a total amount of 0 to 40 wt. %, more preferably 0 to 20 wt. %, more preferably 0.1 to 15 wt. %, and particularly 0.1 to 10 wt. %, based on the total weight of the composition.

Preferable non-ionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk (en) yl oligoglycosides or glucuronic acid derivatives, fatty acid-N-alkyl glucamides, protein hydrolysates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution.

Zwitterionic surfactants are surface-active compounds which contain at least one quaternary ammonium group and at least one COO(−) or SO₃(−) group in the molecule.

Particularly suitable zwitterionic surfactants are the so-called betaines, such as the N-alkyl-N,N-dimethyl ammonium glycinates, for example, cocoalkyl dimethyl ammonium glycinate, N-acylaminopropyl-N,N-dimethyl ammonium glycinates, for example, cocoacylaminopropyl dimethyl ammonium glycinate, and 2-alkyl-3-carboxymethyl-3-hydroxyethyl imidazolines, containing 8 to 18 carbon atoms in the alkyl or acyl group, and cocoacylaminoethyl hydroxyethyl carboxymethyl glycinate. The fatty acid amide derivative known under the CTFA name of Cocamidopropyl Betaine is particularly preferred.

Ampholytic surfactants are also suitable, particularly as co-surfactants. Ampholytic surfactants are surface-active compounds which, in addition to a C₈ to C₁₈ alkyl or acyl group, contain at least one free amino group and at least one —COOH or —SO₃H group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalk-ylaminopropionate, cocoacylaminoethyl aminopropionate and acyl sarcosine.

Anionic surfactants are characterized by a water-solubilizing anionic group such as, for example, a carboxylate, sulfate, sulfonate or phosphate group and a lipophilic group. Dermatologically safe anionic surfactants are known to the practitioner in large numbers from relevant textbooks and are commercially available. They are, in particular, alkyl sulfates in the form of their alkali metal, ammonium or alkanolammonium salts, alkylether sulfates, alkylether carboxylates, acyl isethionates, acyl sarcosinates, acyl taurines containing linear C₁₂-C₁₈ alkyl or acyl groups and sulfosuccinates and acyl glutamates in the form of their alkali metal or ammonium salts.

Particularly suitable cationic surfactants are quaternary ammonium compounds, preferably ammonium halides, more especially chlorides and bromides, such as alkyl trimethyl ammonium chlorides, dialkyl dimethyl ammonium chlorides and trialkyl methyl ammonium chlorides, for example, cetyl trimethyl ammonium chloride, stearyl trim ethyl ammonium chloride, distearyl dimethyl ammonium chloride, lauryl dimethyl ammonium chloride, lauryl dimethyl benzyl ammonium chloride and tricetyl methyl ammonium chloride. In addition, the readily biodegradable quaternary ester compounds, such as, for example, the dialkyl ammonium methosulfates and methyl hydroxyalkyl dialkoyloxyalkyl ammonium methosulfates marketed under the name of Stepantexe and the corresponding products of the Dehyquart® series, may be used as cationic surfactants. “Esterquats” are generally understood to be quaternized fatty acid triethanolamine ester salts. They can provide the compositions with particular softness. They are known substances which are prepared by the relevant methods of organic chemistry. Other cationic surfactants suitable for use in accordance with the invention are the quaternized protein hydrolysates.

Due to the characteristic sensory property of the compound of formula (I) and its substantivity, tenacity as well as stability, it can especially be used to provide an odor, preferably a fragrance impression to surfactant-containing compositions such as, for example, cleaners (in particular laundry care products and all-purpose cleaners). It can preferably be used to impart a long-lasting woody note, fruity note, herbal note, green note, rooty note, citrus note, petitgrain note, floral note, damascone note, tobacco note, ashtray note, gourmand note, sweet note, ionone note, ambra note, fresh note, natural note, Fir Needle note, camphoraceous note, vetiver note to a surfactant comprising composition.

In a preferred embodiment, the composition comprises at least one compound of formula (I) and at least one surfactant and optionally at least one aroma chemical (X).

In another preferred embodiment, the composition comprises at least one compound of formula (Ia), at least one compound of formula (Ib), and at least one compound of formula (Ic) and at least one surfactant and optionally at least one aroma chemical (X).

Suitable compositions are for example perfume compositions, body care compositions (including cosmetic compositions and products for oral and dental hygiene), hygiene articles, cleaning compositions (including dishwashing compositions), textile detergent compositions, compositions for scent dispensers, foods, food supplements, pharmaceutical compositions and crop protection compositions.

Perfume compositions can be selected from fine fragrances, air fresheners in liquid form, gel-like form or a form applied to a solid carrier, aerosol sprays, scented cleaners, perfume candles and oils, such as lamp oils or oils for massage.

Examples for fine fragrances are perfume extracts, Eau de Parfums, Eau de Toilettes, Eau de Colognes, Eau de Solide and Extrait Parfum.

Body care compositions include cosmetic compositions and products for oral and dental hygiene, and can be selected from after-shaves, pre-shave products, splash colognes, solid and liquid soaps, shower gels, shampoos, shaving soaps, shaving foams, bath oils, cosmetic emulsions of the oil-in-water type, of the water-in-oil type and of the water-in-oil-in-water type, such as e.g. skin creams and lotions, face creams and lotions, sunscreen creams and lotions, after-sun creams and lotions, hand creams and lotions, foot creams and lotions, hair removal creams and lotions, after-shave creams and lotions, tanning creams and lotions, hair care products such as e.g. hairsprays, hair gels, setting hair lotions, hair conditioners, hair shampoo, permanent and semi-permanent hair colorants, hair shaping compositions such as cold waves and hair smoothing compositions, hair tonics, hair creams and hair lotions, deodorants and antiperspirants such as e.g. underarm sprays, roll-ons, deodorant sticks and deodorant creams, products of decorative cosmetics such as e.g. eye-liners, eye-shadows, nail varnishes, make-ups, lipsticks and mascara, and products for oral and dental hygiene, such as toothpaste, dental floss, mouth wash, breath fresheners, dental foam, dental gels and dental strips.

Hygiene articles can be selected from joss sticks, insecticides, repellents, propellants, rust removers, perfumed freshening wipes, armpit pads, baby diapers, sanitary towels, toilet paper, cosmetic wipes, pocket tissues, dishwasher and deodorizer.

Cleaning compositions, such as e.g. cleaners for solid surfaces, can be selected from perfumed acidic, alkaline and neutral cleaners, such as e.g. floor cleaners, window cleaners, dishwashing compositions both for handwashing and machine washing use, bath and sanitary cleaners, scouring milk, solid and liquid toilet cleaners, powder and foam carpet cleaners, waxes and polishes such as furniture polishes, floor waxes, shoe creams, disinfectants, surface disinfectants and sanitary cleaners, brake cleaners, pipe cleaners, limescale removers, grill and oven cleaners, algae and moss removers, mold removers, facade cleaners.

Textile detergent compositions can be selected from liquid detergents, powder detergents, laundry pre-treatments such as bleaches, soaking agents and stain removers, fabric softeners, washing soaps, washing tablets.

Food means a raw, cooked, or processed edible substance, ice, beverage or ingredient used or intended for use in whole or in part for human consumption, or chewing gum, gummies, jellies, and confectionaries.

A food supplement is a product intended for ingestion that contains a dietary ingredient intended to add further nutritional value to the diet. A dietary ingredient may be one, or any combination, of the following substances: a vitamin, a mineral, an herb or other botanical, an amino acid, a dietary substance for use by people to supplement the diet by increasing the total dietary intake, a concentrate, metabolite, constituent, or extract. Food supplements may be found in many forms such as tablets, capsules, soft gels, gel caps, liquids, or powders.

Pharmaceutical compositions comprise compositions which are intended for use in the diagnosis, cure, mitigation, treatment, or prevention of disease as well as articles (other than food) intended to affect the structure or any function of the body of man or other animals.

Crop protection compositions comprise compositions which are intended for the managing of plant diseases, weeds and other pests (both vertebrate and invertebrate) that damage agricultural crops and forestry.

Preferably, the compositions according to the invention further comprises at least one auxiliary agent selected from the group consisting of preservatives, abrasives, anti-acne agents, agents to combat skin aging, anti-cellulite agents, antidandruff agents, anti-inflammatory agents, irritation-preventing agents, irritation-alleviating agents, astringents, sweat-inhibiting agents, antiseptics, anti-statics, binders, buffers, carrier materials, chelating agents, cell stimulants, care agents, hair removal agents, emulsifiers, enzymes, essential oils, fibers, film formers, fixatives, foam formers, foam stabilizers, substances for preventing foaming, foam boosters, fungicides, gelling agents, gel-forming agents, hair care agents, hair shaping agents, hair smoothing agents, moisture-donating agents, moisturizing substances, humectant substances, bleaching agents, strengthening agents, stain removal agents, optical brighteners, impregnating agents, soil repellents, friction-reducing agents, lubricants, moisturizing creams, ointments, opacifiers, plasticizers, covering agents, polish, shine agents, polymers, powders, proteins, refatting agents, exfoliating agents, silicones, skin-calming agents, skin-cleansing agents, skin care agents, skin-healing agents, skin lightening agents, skin-protective agents, skin-softening agents, cooling agents, skin-cooling agents, warming agents, skin-warming agents, stabilizers, UV-absorbent agents, UV filters, fabric softeners, suspending agents, skin-tanning agents, thickeners, vitamins, waxes, fats, phospholipids, saturated fatty acids, mono or polyunsaturated fatty acids, alpha hydroxy acids, polyhydroxy fatty acids, liquefiers, dyes, color-protection agents, pigments, anti-corrosives, polyols, electrolytes and silicone derivatives.

Preparation of compositions and methods to impart an aroma impression to a composition.

One embodiment of the present invention is directed to a method for preparing a composition of compound of formula (I) comprising:
(i) at least one aroma chemical (X) other than compounds according to the present invention or
(ii) at least one non-aroma chemical carrier, or both of (i) and (ii).

The invention is also directed to a method for boosting the aroma impression of a composition, wherein the method comprises incorporating at least one compound of formula (I) into a composition.

In particular, the invention is directed to a method of preparing a perfume composition, body care composition, hygiene article, cleaning composition, textile detergent composition, composition for scent dispensers, food, food supplement, pharmaceutical composition or crop protection composition, comprising including at least one compound of formula (I) in a perfume composition, body care composition, hygiene article, cleaning composition, textile detergent composition, composition for scent dispensers, food, food supplement, pharmaceutical composition or crop protection composition.

In one embodiment the invention is directed to a method for imparting a note reminiscent of woody note, fruity note, herbal note, green note, rooty note, citrus note, petitgrain note, floral note, damascone note, tobacco note, ashtray note, gourmand note, sweet note, vetiver note, ionone note, ambra note, fresh note, natural note, Fir Needle note, camphoraceous note elements to a perfume composition, body care composition, hygiene article, cleaning composition, textile detergent composition, composition for scent dispensers, food, food supplement, pharmaceutical composition or crop protection composition, which comprises including at least one compound of formula (I) in a perfume composition, body care composition, hygiene article, cleaning composition, textile detergent composition, composition for scent dispensers, food, food supplement, pharmaceutical composition or crop protection composition.

### Amounts

Generally, the total amount of compounds of formula (I) in the compositions, methods and uses according to the present invention is typically adapted to the particular intended use or the intended application and can, thus, vary over a wide range. As a rule, the customary standard commercial amounts for aroma chemicals, preferably for scents are used.

Preferably, the compositions according to the invention comprise compounds of formula (I) in a total amount of 0.001 to 99.9 wt. %, based on the total weight of the composition.

Particularly, the compositions comprise compounds of formula (I) in a total amount of 0.001 to 99.5 wt. %, preferably of 50 to 99 wt. %, more preferably of 80 to 95 wt. % and in particular of 90 to 95 wt. %, based on the total weight of the composition.

Particularly, the compositions comprise compounds of formula (I) in a total amount of 0.005 to 80 wt. %, preferably of 0.1 to 30 wt. %, more preferably of 1 to 20 wt. %, and in particular of 5 to 15 wt. %, based on the total weight of the composition.

Particularly, the compositions comprise the compounds of formula (I) in a total amount of 0.001 to 20 wt. %, preferably of 0.005 to 6 wt. %, more preferably of 0.05 to 4 wt. %, and in particular of 0.1 to 3 wt. %, based on the total weight of the composition.

The terms compound (I) and compound of formula (I) are used interchangeably throughout the specification. Also, the term compounds (I) and compounds of formula (I) are used interchangeably throughout the specification.

### Embodiments

In the following, there is provided a list of embodiments to further illustrate the present disclosure without intending to limit the disclosure to the specific embodiments listed below.
1. Use of at least one compound of formula (I)

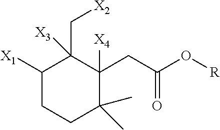

compound of formula (I)
or stereoisomers thereof,
where,
X₁ and X₃ together form a double bond between the carbon atoms to which they are bound, with the proviso that X₂ and X₄ are hydrogen; or
X₃ and X₄ together form a double bond between the carbon atoms to which they are bound, with the proviso that X₁ and X₂ are hydrogen; or
X₂ and X₃ together form a double bond between the carbon atoms to which they are bound, with the proviso that X₁ and X₄ are hydrogen, or
X₄ is OH, X₁, X₂ and X₃ are all hydrogen,
where R is selected from C₁-C₅ linear or branched alkyl and C₃-C₅ linear or branched alkenyl,
to impart an aroma impression to a composition.
2. The use according to embodiment 1, wherein compound of formula (I) comprises at least one compound of formula (Ia)

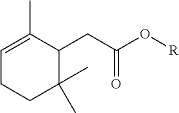

compound of formula (Ia)
or its stereoisomers or mix of its stereoisomers,
or at least one Compound of formula (Ib),

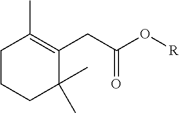

compound of formula (Ib)
or at least one compound of formula (Ic),

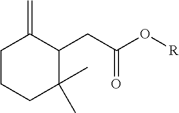

compound of formula (Ic)
or its stereoisomers or mix of its stereoisomers,
or at least one compound of formula (Id),

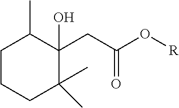

compound of formula (Id)
or its stereoisomers or mix of its stereoisomers,
or mixtures thereof;
where,
R is selected from C₁-C₅ linear or branched alkyl and C₃-C₅ linear or branched alkenyl.
3. The use according to any of the embodiments 1 to 2, wherein R is selected from methyl, ethyl, propyl, butyl, isobutyl, isopropyl, 1-propenyl, or 2-propenyl.
4. The use according to any of the embodiments 1 to 3, wherein R is selected from methyl ethyl or 1-propenyl.
5. The use of the mixture according to embodiments 2 to 4, wherein the weight ratio of the at least one compound of formula (Ia) and at least one compound of formula (Ib) is in the range of 0.1:99.9 to 99.9:0.1.
6. The use of the mixture according to any of the embodiments 2 to 5, wherein the weight ratio of the at least one compound of formula (Ia) and at least one compound of formula (Ib) is in the range of 20:80 to 80:20, preferably 30:70 to 70:30 or preferably 40:60 to 60:40 or preferably 50:50.
7. The use of the mixture according to any of the embodiments 2 to 6, wherein the mixture has a ratio in the range of 90:10 to 99.9:0.1, wherein the ratio represents the combined amount of compound of formula (Ia) and compound of formula (Ib) to the compound of formula (Ic).
8. A method of imparting an aroma impression to a composition, comprising at least the step of adding the compound of formula (I) according to any of the embodiments 1 to 7 to a composition.
9. The use or method according to any of the embodiments 1 to 8, wherein the aroma impression is selected from the group consisting of woody note, fruity note, herbal note, green note, rooty note, citrus note, petitgrain note, floral note, damascone note, tobacco note, ashtray note, gourmand note, sweet note, vetiver note, ionone note, ambra note, fresh note, natural note, Fir Needle note, camphoraceous note, or any combination of two or more of these notes.
10. The use or method according to any of the embodiments 1 to 8, wherein the at least one compound of formula (I) is used in a total amount in the range of ≥0.001 wt. % to ≤70.0 wt. %, based on the total weight of the composition.
11. A composition comprising,
   i) the at least one compound of formula (I) according to any of the embodiments 1 to 7, and
   ii) at least one aroma chemical (X) other than compounds of formula (I), or
   iii) at least one non-aroma chemical carrier, or
   iv) both of (ii) and (iii).
12. The composition according to embodiment 11, wherein the at least one compound according to embodiment 1 to 7 is present in the range of ≥0.01 wt. % to ≤70.0 wt. %, based on the total weight of the composition.
13. The composition according to embodiment 11, wherein the at least one non-aroma chemical carrier (ii) is selected from surfactants, oil components, antioxidants, deodorant-active agents, or solvents.
14. The composition according to any of the embodiments 11 to 13, wherein the composition is selected from perfume compositions, body care compositions, hygiene articles, cleaning compositions, textile detergent compositions, compositions for scent dispensers, foods, food supplements, pharmaceutical compositions, or crop protection compositions.
15. A mixture comprising at least one compound of formula (Ib).

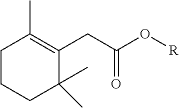

compound of formula (Ib),
and
at least one compound of formula (IV)

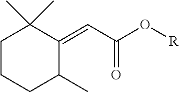

compound of formula (IV)
or its stereoisomers or mix of its stereoisomers where,
R is selected from C₁-C₅ linear or branched alkyl and C₃-C₅ linear or branched alkenyl.
16. Use of a mixture according to embodiment 15, to impart a impart an aroma impression to a composition.
17. A method of imparting an aroma impression to a composition comprising at least the step of adding the mixture according to embodiment 15 to a composition.
18. The mixture, or the use, or the method according any of the embodiment 15 to 17, wherein the weight ratio of the of compound of formula (Ib) and (IV) is in the range 90:10 to 99:1.
19. The use or method according to any of the embodiments 16 to 18, wherein the aroma impression is selected from leather or plastic or a combination thereof.
20. A process for preparing the mixture of at least one compound of formula (I) according to any of the embodiments 1 to 7, comprising at least the step of a) Cyclization of the compound of formula (III)

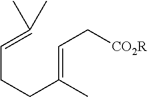

compound of formula (III)
where R is selected from C₁-C₅ linear or branched alkyl and C₃-C₅ linear or branched alkenyl,
using catalytic amount of acid to produce the at least one compound of formula (I), b) optionally purifying the at least one compound of formula (I).

### EXAMPLES

The present invention is illustrated in detail by non-restrictive working examples which follow. More particularly, the test methods specified hereinafter are part of the general disclosure of the application and are not restricted to the specific working examples.

1. Analytical method and Materials:

### Materials:

### Chemicals:

Chemicals were purchased from commercial vendors (ABCR, Acros Organics, Alfa Aesar, Apollo Scientific, Fluorochem, Manchester Organics, Sigma-Aldrich, TCI) and used without further purification unless otherwise noted.

Analytical methods:
NMR spectroscopy:

The characterization is done by 13C NMR and 1H NMR. The 13C NMR and 1H NMR spectra were measured on a Bruker AV-500 spectrometer.

### (Flash) Column Chromatography

(Flash) Column chromatography was performed using silica gel (60 Å, 230-400 mesh, particle size: 43-63 μm) from Merck or using distilled technical grade solvents. The solvent mixtures and volume ratios (v/v) used as mobile phase for chromatography are specified in the corresponding experiment. Flash column chromatography was performed in glass columns by applying slightly elevated air or argon (0.3 mbar) pressure.

### Gas Chromatography (GC)

Gas Chromatography (GC) was performed on HP 6890 and 5890 Series instruments equipped with a split-mode capillary injection system and a flame ionization detector (FID) using hydrogen (H₂) as carrier gas.

### 2. Synthesis

### Example 1: Preparation of alpha-Methyl cyclohomogerate (a-1), beta-Methyl cyclohomogerate (β-1), gamma-Methyl cyclohomogerate (v-1)

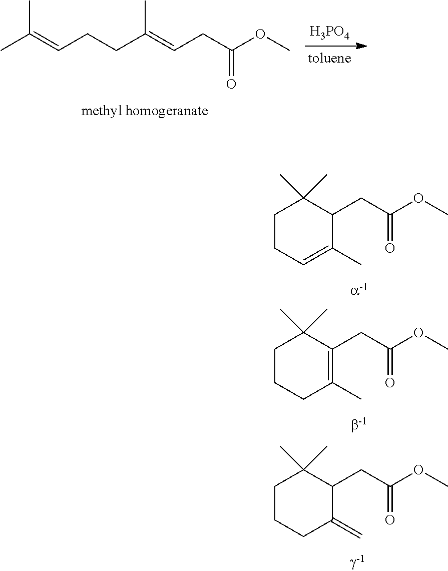

In a 250 ml reactor (equipped with flow breakers, impeller stirrer, condenser and Ar-inert gas connection) methyl homogeranate (53 g, sum E/Z-isomers 97.65%, 0.264 mol) was dissolved in toluene (120 ml) and heated to 100° C. The solution was stirred vigorously and a catalytic amount of H₃PO₄ (85%, 4.4 g) was added via a syringe. The reaction mixture was stirred at 100° C. for 2 h. Reaction control via gas chromatography indicated a full conversion of the methyl homogeranate. The reaction mixture was cooled to room temperature and quenched with sat. Na₂CO₃-solution (250 ml). The organic layer was separated from the aqueous layer and the aqueous layer was extracted two times with 100 ml methyl-tert-butylether (MTBE). The combined organic phase was dried over Na₂SO₄, filtered and the solvents were removed via rotary evaporator in vacuum (100-10 mbar) at 40-50° C. After the solvent was removed, 52 g of a yellowish residue with the following product distribution was obtained via GC: 36.3% alpha-cyclohomogeranic acid methylester (α-1), 35.7% beta-cyclohomogeranic acid methylester (β-1), 2.9% gamma-cyclohomogeranic acid methylester (γ-1).

44 g of the crude product were purified in a fractional batch distillation (concentric tube column, 60 theor. number of plates) at a pressure of 1 mbar, a sump temperature of 90° C. to 112° C. and a reflux ratio of 50:1. The content and purity of the different fractions are summarized in table 1.

TABLE 1
Fractional distillation of cyclohomogeranic acid methylester (1)

| Mass | | Fraction | α-1 [GC-%] | β-1 [GC-%] | γ-1 [GC-%] |
|---|---|---|---|---|---|
| 1.6 | g | FR. 1 | 42.3 | 48.2 | 2.7 |
| 1.3 | g | FR. 2 | 42.5 | 48.4 | 2.7 |
| 0.6 | g | FR. 3 | 41.2 | 48.2 | 2.6 |
| 1.9 | g | FR. 4 | 39.8 | 48.3 | 2.7 |
| 0.9 | g | FR. 5 | 44.1 | 48.1 | 2.8 |
| 1.75 | g | FR. 6 | 80.6 | 18.4 | 0.9 |
| 1.63 | g | FR. 7 | 70.7 | 27.8 | 1.5 |
| 0.72 | g | FR. 8 | 41.9 | 54.6 | 3.4 |
| 0.65 | g | FR. 9 | 16.7 | 78.0 | 5.3 |
| 0.48 | g | FR. 10 | 2.6 | 91.6 | 5.8 |
| 0.75 | g | FR. 11 | 0.90 | 91.8 | 4.5 |
| 0.88 | g | FR. 12 | 4.3 | 9.0 | 0.5 |
| 6.54 | g | Sump | 0.00 | 0.10 | 0.0 |
| 15.9 | g | Cooling trap | | | |

The distillation fractions were used to obtain the fragrance samples in table 2.

TABLE 2
Composition of the fractions (enriched isomers) selected
for olfactory evaluation as per example 1

| Fraction no. | α-1 [GC-%] | β-1 [GC-%] | γ-1 [GC-%] | sample no |
|---|---|---|---|---|
| Fraction 3 | 42.5 | 48.4 | 2.7 | S1 |
| Fraction 6 | 80.6 | 18.4 | 0.9 | S2 |
| Fraction 11 | 0.9 | 91.8 | 4.5 | S3 |

### Example 2: Preparation of alpha-cyclohomogeranic acid ethylester (α-2), beta-cyclohomogeranic acid ethylester (β-2), gamma-cyclohomogeranic acid ethylester (γ-2)

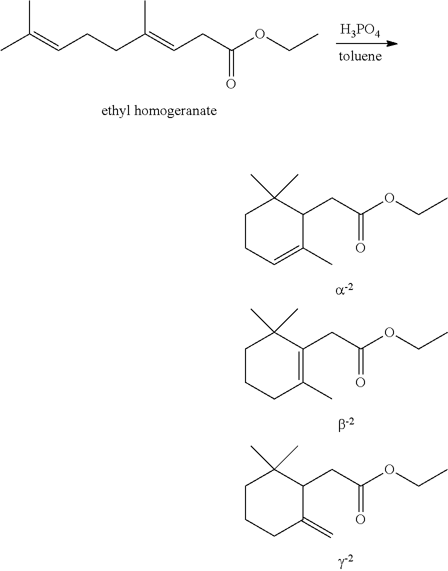

In a 500 ml reactor (equipped with flow breakers, impeller stirrer, condenser and Ar-inert gas connection) the starting material ethyl homogeranate (120 g, sum E/Z-isomers 97.5%, 0.264 mol) was dissolved in toluene (350 ml) and heated to 100° C. The solution was stirred vigorously and a catalytic amount of H₃PO₄ (85%, 10.2 g) was added via a syringe. The reaction mixture was stirred at 100° C. for 4 h. Reaction control via gas chromatography indicated a conversion of the ethyl homogeranate of >90%. The reaction mixture was cooled to room temperature and quenched with sat. Na₂CO₃-solution (400 ml). The organic layer was separated from the aqueous layer and the aqueous layer was extracted two times with 250 ml methyl-tert-butylether (MTBE). The combined organic phased were dried over Na₂SO₄, filtered and the solvents were removed via rotary evaporator in vacuum (100-10 mbar) at 40-50° C. After the removal of the solvent, 114 g of a yellowish residue with the following product content was obtained: GC-analysis α-2+β-2+γ-2=68.9% (no separation of isomers via GC). 114 g of the crude product was purified in a fractional batch distillation (concentric tube column, 60 theor. number of plates) at a pressure of 1 mbar, a sump temperature of 90° C. to 93° C. and a reflux ratio of 50:1. The content and purity of the different fractions are summarized in table 3.

TABLE 3
Fractional distillation of cyclohomogeranic acid ethylester (2)

| | | | GC-content impurity | GC-content |
|---|---|---|---|---|
| Mass | | Fraction | light boiler [%] | α-2 + β-2 + γ-2 [%] |
| 2.2 | g | FR. 1 | 14.72% | 84.4% |
| 2.2 | g | FR. 2 | 1.47% | 98.1% |
| 2.5 | g | FR. 3 | 0.54% | 99.0% |
| 2.83 | g | FR. 4 | 0.23% | 99.3% |
| 7.47 | g | FR. 5 | 0.09% | 99.4% |
| 2.20 | g | FR. 6 | 0.11% | 99.3% |
| 2.20 | g | FR. 7 | 0.02% | 99.4% |
| 2.70 | g | FR. 8 | 0.04% | 99.3% |
| 2.20 | g | FR. 9 | 0.11% | 99.2% |
| 2.20 | g | FR. 10 | 0.02% | 99.3% |
| 4.70 | g | FR. 11 | 0.26% | 99.2% |
| 3.20 | g | FR. 12 | 0.04% | 99.9% |
| 3.20 | g | FR. 13 | — | 98.9% |
| 2.90 | g | FR. 14 | — | 98.9% |
| 1.95 | g | FR. 15 | 0.09% | 98.7% |
| 3.95 | g | FR. 16 | 0% | 98.6% |
| 0.78 | g | FR. 17 | — | 98.1% |
| 37.31 | g | Sump | 0.39% | 12.0% |
| 14.2 | g | cooling trap | | |

Analysis via NMR indicated only minor separation of the isomers. Therefore, the following fractions were unified to three fragrance samples-see table 4.

NMR-analysis of FR. 4-8

α/β-cyclohomogeranic acid ethylester (α/β-2)

NMR data of distillation fraction FR.4-8 showed a ratio α-2:β-2=45:55 13C NMR (CDCl₃, 126 MHz) δ (ppm) 174.4, 172.8, 135.2, 131.3, 130.5, 121.35, 60.4, 60.3, 45.6, 39.3, 35.9, 34.7, 33.8, 32.7, 32.2, 31.3, 28.1, 27.9, 26.9, 26.2, 22.9, 22.7, 20.3, 19.4, 14.2, 14.2.

TABLE 4
Composition of the fractions (enriched isomers) selected
for olfactory evaluation as per example 2

| Fraction | α-2 | β-2 | γ-2 | |
|---|---|---|---|---|
| no. | [NMR-%] | [NMR-%] | [NMR-%] | Sample no. |
| FR. 4-8 | 45 | 55 | <1 | S4 |
| FR. 9-12 | 40 | 60 | <1 | S5 |
| FR. 13-17 | 28 | 72 | <1 | S6 |

### Example 3: Preparation of alpha-cyclohomogeranic acid methylester (α-1), beta-cyclohomogeranic acid methylester (β-1), gamma-cyclohomogeranic acid methylester (γ-1)

A 100 mL round-bottom flask was charged with (E/Z)-homogeranic acid methyl ester (3.92 g, 20 mmol) and a PTFE-coated magnetic stir bar. The starting material was dissolved in hexafluoroisopropanol (20 mL, 9.5 equiv., 1 M) and TFA (trifluoroacetic acid, 2.38 g, 20.9 mmol) was added dropwise (an immediate color change from colorless over bright yellow then bright orange to dark orange-red was observed upon addition of TFA within 1 min) to the stirred solution and the resulting red solution was stirred at 25° C. for 24 h. After the elapsed time, the dark brown-red reaction mixture was concentrated under reduced pressure. The residue was dissolved in hexanes/MTBE (19:1 v/v, 20 mL), Celite was added and the slurry was concentrated under reduced pressure. Purification by flash column chromatography on silica gel (Merck, 40-63 μm, 300 g, d×I=6×22 cm) using hexanes/MTBE (gradient elution: hexanes/MTBE=19:1 to 4:1 v/v) as eluent followed by drying in vacuo overnight afforded cyclohomogeranic acid methylester as a colorless oil (2.34 g, 11.9 mmol, 60% yield, with α-1:β-1: γ-1=31:68:1 based on GC). The purified mixture of isomers was used as fragrance samples (table 5).

TABLE 5
Composition of the fractions (enriched isomers) selected
for olfactory evaluation as per example 3

| Fraction | α-1 [GC-%] | β-1 [GC-%] | γ-1 [GC-%] | Sample no. |
|---|---|---|---|---|
| EXP3 | 31 | 68 | 1 | S7 |

### Example 4: Preparation of gamma-cyclohomogeranic acid methylester (v-1)

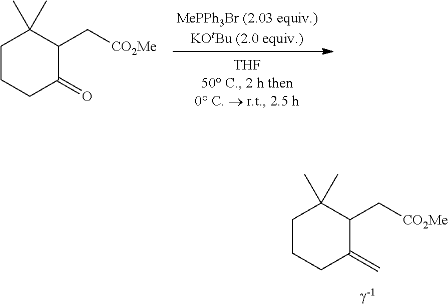

A 50 mL Schlenk tube equipped with a PTFE-coated magnetic stir bar under argon was charged with methyltriphenylphosphonium bromide (3.60 g, 10.1 mmol) and potassium tert-butoxide (1.1 g, 9.80 mmol). Anhydrous THF (25 mL) was added, the Schlenk tube was immersed in a preheated oil bath at 50° C. and the resulting bright yellow suspension was heated at this temperature for 2 h. After this time, the yellow reaction mixture was cooled to 0° C., and a solution of methyl 2-(2,2-dimethyl-6-oxocyclohexyl) acetate (0.98 g, 4.97 mmol) in dry THF (7 mL) was added dropwise. After the addition was complete, the resulting orange-brown reaction mixture was stirred at room temperature for 2.5 h. After this time, the reaction mixture was treated with water (50 mL) and brine (50 mL), and the aqueous phase was extracted with diethyl ether (3×50 mL). The combined organic layers were dried over Na₂SO₄, filtered, and concentrated under reduced pressure to provide the crude product as a yellow oil. Purification by flash column chromatography on silica gel using hexanes/MTBE (gradient elution: hexanes/MTBE 39:1 to 19:1 v/v) as eluent furnished (±)-γ-cyclohomogeranic acid methylester (γ-1), 753 mg, 3.84 mmol, 77% yield) as a colorless oil.

γ-cyclohomogeranic acid methylester (v-1)

¹H NMR (CDCl₃, 501 MHZ) δ (ppm)=4.75 (q, J=1.4 Hz, 1H), 4.56 (s, 1H), 3.64 (s, 3H), 2.50 (t, J=10.2 Hz, 1H), 2.46-2.38 (m, 2H), 2.22 (dt, J=12.7, 6.1 Hz, 1H), 2.05 (ddd, J=13.1, 8.2, 5.3 Hz, 1H), 1.43 (ddd, J=11.6, 7.2, 4.3 Hz, 1H), 1.35 (ddd, J=13.3, 8.1, 4.9 Hz, 1H), 0.96 (s, 3H), 0.79 (s, 3H).

GC DB-Waxetr 0.25 mm/0.25 mm, 30 m, temperature: 220° C. (injector)/from 60° C. to 135° C. with 2° C./min, then with 6° C./min to 220° C., then with 12° C./min to 260° C., then 5 min at 260° C., 350° C. (detector), gas: 0.60 bar H₂, sample size: 0.2 μL, t_{R}=29.71 min (99%).

The so obtained gamma-cyclohomogeranic acid methylester (y-1) was evaluated for its aroma impression. This was labelled as sample S8.

### Example 5: Preparation of beta-cyclohomogeranic acid methylester (β-1)

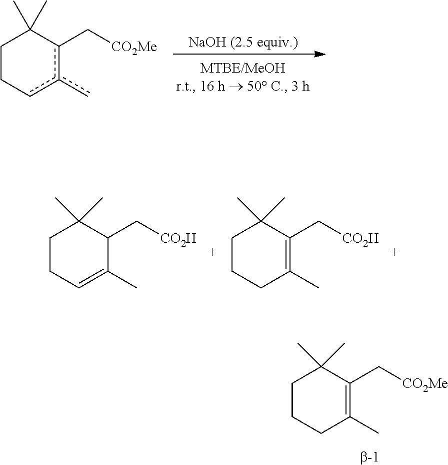

Sodium hydroxide (powder; 1.53 g, 38.3 mmol, 2.5 equiv.) was transferred to a 50 mL round-bottom flask equipped with a PTFE-coated magnetic stir bar under an atmosphere of Argon.Methanol and MTBE (15 mL each) were added and the resulting colorless suspension was stirred until the sodium hydroxide was nearly completely dissolved. The methyl ester (mixture of α-1/β-1/γ-1; 3.00 g, 15.3 mmol, 1.0 equiv.) was added via syringe and the resulting pale yellow solution was stirred at room temperature overnight. After the elapsed time, 1H NMR analysis of the crude reaction mixture indicated an approximate ratio of 25:75 a-1:3-1 of remaining methyl cyclohomogeranates). After heating to 50° C. for 3 h, 1H NMR analysis indicated an approximate ratio of 10:90 a-1:3-1 of the remaining methyl cyclohomogeranates. The reaction mixture was concentrated under reduced pressure, diluted with water (20 mL) and MTBE (20 mL). The biphasic mixture was poured into ice-cold 10% aqueous hydrochloric acid (15 mL) and water (20 mL). The phases were separated and the aqueous phase was extracted with MTBE (3×50 mL). The combined organic layers were washed with brine (50 mL), dried over Na₂SO₄, filtered and concentrated under reduced pressure. 1H NMR analysis of the crude reaction mixture indicated a-1:3−1=3:97. The ratio between B-1 and B-cyclohomogeranic acid was 54:46. Purification of the crude reaction mixture by flash column chromatography on silica gel (200 g, d×I=6×15 cm) eluting with hexanes/MTBE (19:1 v/v) afforded β-1 as a colorless oil (799 mg, 4.07 mmol, 27% yield). Further elution with hexanes/ethyl acetate (gradient elution: 4:1 to 2:1 v/v) afforded a mixture of α- and β-cyclohomogeranic acids as a colorless solid (1.43 g, 7.82 mmol, 51% yield).

β-cyclohomogeranic acid methylester (β-1)

¹H NMR (CDCl₃, 501 MHZ) δ (ppm)=3.66 (s, 3H), 3.05 (s, 2H), 2.01-1.96 (m, 2H), 1.65-1.54 (m, 2H), 1.58 (s, 3H), 1.48-1.44 (m, 2H), 0.96 (s, 6H).

¹³C NMR (CDCl₃, 126 MHZ) δ (ppm)=173.5, 131.6, 130.5, 51.8, 39.5, 34.9, 33.7, 32.8, 28.1, 20.4, 19.5.

GC DB-Waxetr 0.25 mm/0.25 mm, 30 m, temperature: 220° C. (injector)/from 60° C. to 135° C. with 2° C./min, then with 6° C./min to 220° C., then with 12° C./min to 260° C., then 5 min at 260° C., 350° C. (detector), gas: 0.60 bar H₂, sample size: 0.2 μL, (R=29.0 min (GC-MS: m/z [M]⁺=196).

HRMS (GC-EI) m/z calculated for C₁₂H₂₀O₂⁺ [M]⁺ 196.1458; found: 196.1457.

The so obtained beta-cyclohomogeranic acid methylester (B-1) was evaluated for its aroma impression. This was labelled as sample S10.

### Example 6: Preparation of methyl (E)-2-(2,2,6-trimethylcyclohexylidene) acetate (3/compound of formula (IV))

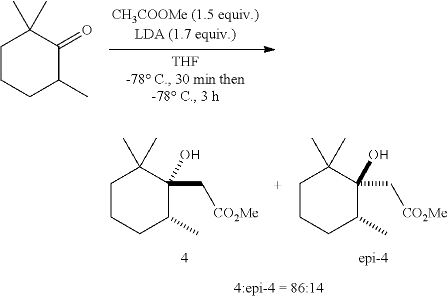

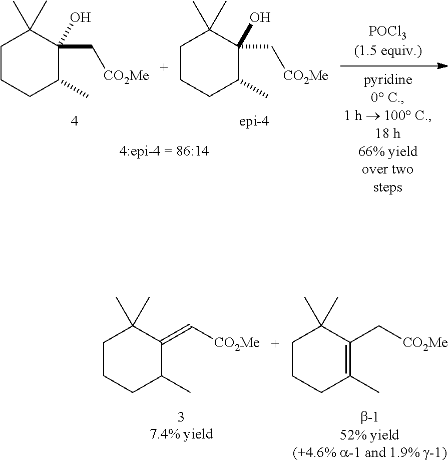

Diisopropylamine (3.25 g, 32.1 mmol, 1.7 equiv.) was transferred to a flame-dried 250 mL round-bottom flask equipped with a PTFE-coated magnetic stir bar and fitted with an argon outlet and a rubber septum. Anhydrous THF (30 mL) was added and the colorless solution was cooled to −78° C. Then, n-butyllithium (2.5 M solution in n-hexane, 12 mL, 30 mmol, 1.6 equiv.) was added and the resulting bright yellow solution was stirred at −78° C. for 15 min. Then anhydrous methyl acetate (2.05 g, 27.6 mmol, 1.5 equiv.) was added dropwise. After completed addition, stirring was continued for 30 min. Then, a solution of 2,6,6-trimethylcyclohexanone (2.62 g, 18.7 mmol, 1.0 equiv.) in anhydrous THF (10 mL, flask was rinsed with 2×5 mL) was added dropwise at −78° C. After the addition was complete, the reaction mixture was stirred at this temperature for further 3 h. Then, aqueous saturated NH₄CI solution (50 mL) was added and the reaction mixture was diluted with MTBE (20 mL) and water (50 mL). The organic phase was separated and the aqueous phase was extracted with MTBE (3×50 mL). The combined organic layers were washed with brine (50 mL), dried over Na₂SO₄, filtered and concentrated under reduced pressure to afford the crude product as a pale yellow oil (4.00 g, 18.7 mmol, 4: epi-4=86:14 mixture of diastereomers). The crude B-hydroxyester was directly used in the next step without further purification. Distilled phosphoryl chloride (2.6 mL, 4.30 g, 28.0 mmol, 1.5 equiv.) was added to a solution of the B-hydroxy ester in anhydrous pyridine (20 mL) at 0° C. and the resulting colorless solution was stirred for 30 min at room temperature. Then, the reaction vessel was immersed in a preheated oil bath at 100° C. and the reaction mixture was stirred at this temperature for 18 h. After cooling to room temperature, the dark brown reaction mixture was quenched with ice-cold water (50 mL), diluted with MTBE (50 mL) and filtered over a small pad of silica gel into separatory funnel containing 100 mL 5% aqueous HCl. The phases were separated and the aqueous phase was extracted with MTBE (3×100 mL). The combined organic layers were washed with 1 M HCl (150 mL), and brine (150 mL), dried over Na₂SO₄, filtered and concentrated under reduced pressure to afford the crude product as a yellow oil. Purification by flash column chromatography on silica gel using hexanes/MTBE (gradient elution: hexanes/MTBE 39:1 to 19:1 v/v) as eluent afforded the title compounds as colorless oils. Methyl (E)-2-(2,2,6-trimethylcyclohexylidene)-acetate 3 was obtained (272 mg, 1.38 mmol, 7.4% yield) besides a-1 (168 mg, 0.86 mmol, 4.6% yield), β-1 (1.91 g, 9.73 mmol, 52% yield), and y-1 (71.3 mg, 0.36 mmol, 1.9% yield), corresponding to an overall yield of 66% over two steps from 2,2,6-trimethylcyclohexanone. NMR data for the methyl cyclohomogeranate isomers were in accordance with literature data.

Methyl (E)-2-(2,2,6-trimethylcyclohexylidene) acetate (3)

¹H NMR (CD₂Cl₂, 501 MHZ) δ (ppm)=5.75 (s, 1H), 4.02-3.89 (m, 1H), 3.64 (s, 3H), 1.82 (qt, J=12.7, 3.5 Hz, 1H), 1.62-1.50 (m, 3H), 1.49-1.42 (m, 1H), 1.39 (td, J=12.8, 4.1 Hz, 1H), 1.22 (d, J=7.5 Hz, 3H), 1.16 (s, 3H), 1.11 (s, 3H).

¹³C NMR (CDCl₃, 126 MHZ) δ (ppm)=176.2, 77.1, 52.2, 40.1, 38.0, 37.8, 32.5, 31.9, 26.1, 22.2, 21.6, 16.2.

HRMS (GC-EI) m/z calculated for C₁₂H₂₀O₂⁺ [M]⁺ 196.1458; found: 196.1458.

GC DB-Waxetr 0.25 mm/0.25 mm, 30 m, temperature: 220° C. (injector)/from 60° C. to 135° C. with 2° C./min, then with 6° C./min to 220° C., then with 12° C./min to 260° C., then 5 min at 260° C., 350° C. (detector), gas: 0.60 bar H₂, sample size: 0.2 μL, (R=31.63 min (99%; GC-MS: m/z [M]⁺=196).

The so obtained methyl (E)-2-(2,2,6-trimethylcyclohexylidene) acetate (3) was evaluated for its aroma impression. This was labelled as sample S11.

### Example 8: Preparation of allyl 2-(2,2,6-trimethylcyclohexy-2-en-1-yl) acetate (a/B-5)

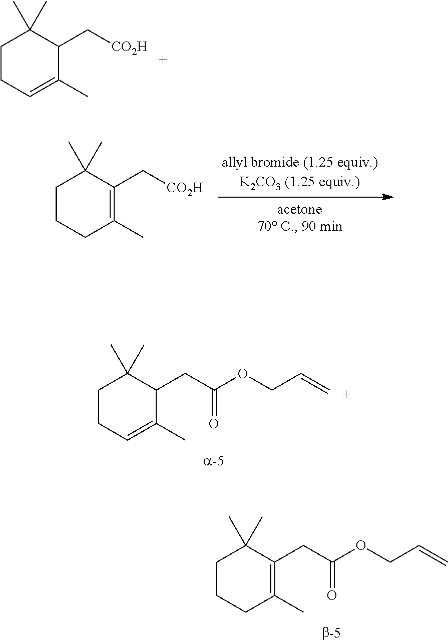 [SMILES: CC1=CCCC(C)(C)C1CC(=O)O ; CC1=C(CC(=O)O)C(C)(C)CCC1 ; C=CCOC(=O)CC1C(C)=CCCC1(C)C ; C=CCOC(=O)CC1=C(C)CCCC1(C)C]

A 20 mL headspace Biotage microwave reactor vial was charged with a PTFE-coated magnetic stir bar and anhydrous K₂CO₃ (256 mg, 1.85 mmol, 1.25 equiv.). The vial was sealed with a rubber septum, evacuated, flame-dried backfilled with argon after cooling to room temperature. The carboxylic acid (mixture of isomers a: β=57:43; 270 mg, 1.48 mmol, 1.0 equiv.) and dry acetone (7.5 mL) were added sequentially. Allyl bromide (224 mg, 1.85 mmol, 1.25 equiv.) was transferred to the stirred colorless suspension. The rubber septum was exchanged with a crimp cap containing a PTFE-silicone septum and sealed. The reaction vessel was immersed in a preheated oil bath at 70° C. and the resulting colorless suspension was vigorously stirred (1400 rpm) at this temperature for 90 min. After cooling to room temperature, the vial was opened, the colorless reaction mixture was diluted with MTBE (5 mL) and filtered over cotton wool onto celite (reaction vial was rinsed with 4×5 mL MTBE). The suspension was concentrated under reduced pressure and directly purified by flash column chromatography on silica gel (50 g, d x | =3×15 cm) using hexanes/MTBE (19:1 v/V) as eluent to afford the desired product as a colorless oil (288 mg, 1.30 mmol, 88% yield; 99% GC purity; a-5:3−5=55:45).

Characterization data for the individual isomers:

α-5:

¹H NMR (501 MHZ, CDCl₃) δ (ppm)=5.93 (ddt, J=17.3, 10.4, 5.8 Hz, 1H), 5.36-5.33 (m, 1H), 5.33 (dq, J=17.0, 1.5 Hz, 1H), 5.24 (dq, J=10.3, 1.3 Hz, 1H), 4.58 (dt, J=6.0, 1.3 Hz, 2H), 2.38 (dd, J=17.4, 8.2 Hz, 1H), 2.27-2.19 (m, 2H), 2.01-1.92 (m, 2H), 1.66 (q, J=1.9 Hz, 3H), 1.42-1.32 (m, 1H), 1.23-1.14 (m, 1H), 0.92 (s, 3H), 0.84 (s, 3H).

¹³C NMR (126 MHZ, CDCl₃) δ (ppm)=174.2, 135.3, 132.4, 121.6, 118.4, 65.3, 45.7, 35.9, 32.4, 31.5, 27.0, 26.4, 23.0, 22.9.

β-5:

1H NMR (501 MHZ, CDCl₃) δ(ppm)=5.92 (ddt, J=17.2, 10.4, 5.7 Hz, 1H), 5.32 (dq, J=17.2, 1.6 Hz, 1H), 5.22 (dq, J=10.4, 1.4 Hz, 1H), 4.57 (dt, J=5.7, 1.4 Hz, 2H), 3.08 (s, 2H), 1.99 (t, J=6.4 Hz, 2H), 1.63-1.56 (m, 2H), 1.59 (s, 3H), 1.48-1.44 (m, 2H), 0.96 (s, 6H). 13C NMR (126 MHZ, CDCl₃): δ(ppm) 172.6, 132.6, 131.7, 130.5, 118.1, 65.2, 39.5, 34.9, 33.9, 32.9, 28.1, 20.5, 19.5.

GC DB-1 0.25 mm/0.25 mm, 30 m, temperature: 220° C. (injector)/from 50° C. to 140° C. with 2° C./min, then with 12° C./min to 350° C., 350° C. (detector), gas: 0.60 bar H₂, split ratio 120:1, sample size: 0.2 ML;® (α-isomer)=41.0 min (54%; GC-MS: m/z [M]⁺=222), t_{R} (β-isomer)=41.3 min (44%; GC-MS: m/z [M]⁺=222); 99% GC purity.

HRMS (GC-EI) m/z calculated for C₁₄H₂₂₀₂+ [M]⁺222.1614; found: 222.1614.

The so obtained allyl 2-(2,2,6-trimethylcyclohexy-2-en-1-yl) acetate (a/B-5) was evaluated for its aroma impression. This was labelled as sample S12.

### Example 9: Preparation of (+)-methyl 1-hydroxy-2,2,6-trimethylcyclohexaneacetate (4)

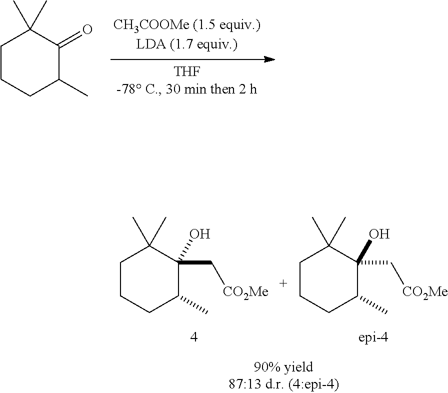

Freshly distilled diisopropylamine (1.59 g, 15.7 mmol, 1.7 equiv.), was transferred to a flame-dried two-neck round-bottom flask equipped with a rubber septum and an argon outlet under argon. Dry THF (15 mL) was added and the flask was immersed in a dry-ice/acetone cooling bath at −78° C. After 10 min, n-butyllithium (2.5 M solution in n-hexane, 4.30 g, 15.5 mmol, 1.7 equiv.) was added dropwise and the resulting yellow solution was stirred at this temperature for 15 min. Subsequently anhydrous methyl acetate (1.02 g, 13.8 mmol, 1.5 equiv.) was added dropwise and stirring was continued for additional 30 min at −78° C. Then, a solution of commercially available 2,2,6-trimethylcyclohexan-1-one (1.29 g, 9.21 mmol, 1.0 equiv.) in THF (10 mL) was added, and the reaction mixture was stirred for further 2 h at −78° C. After the elapsed time, aqueous saturated ammonium chloride solution (20 mL) was added, followed by water (20 mL) and MTBE. (20 mL). The organic phase was separated, and the aqueous phase was extracted with MTBE (3× 20 mL). The combined organic layers were washed with brine (50 mL), dried over Na₂SO₄, filtered and concentrated under reduced pressure to give the crude product as a pale yellow oil as a mixture of diastereomers. Purification by flash column chromatography on silica gel (gradient elution with hexanes/MTBE 39:1 to 19:1 v/v) afforded diastereomerically pure equatorial isomer 4 (912 mg, 4.26 mmol, 46% yield) as a colorless oil besides mixed fractions (863 mg, 4.03 mmol, 44% yield) with 4: epi-4=2.7:1. This corresponds to a yield of 78% for 4 and 12% of epi-4 resulting in an overall yield of 90%. The so obtained 4 was evaluated for its aroma impression. This was labelled as sample S13.

Characterization data for the major diastereomer 4:

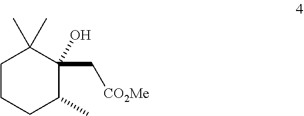

¹H NMR (CD₂Cl₂, 600 MHZ) δ (ppm)=4.17 (s, 1H), 3.67 (s, 3H), 2.54 (d, J=15.7 Hz, 1H), 2.37 (d, J=15.7 Hz, 1H), 1.79-1.70 (m, 2H), 1.54-1.38 (m, 3H), 1.32-1.27 (m, 1H), 1.06-1.00 (m, 1H), 0.93 (s, 3H), 0.90 (s, 3H), 0.81 (d, J=6.7 Hz, 3H).

¹³C NMR (CD₂Cl₂, 151 MHZ) δ (ppm)=175.9, 76.2, 52.2, 39.3, 37.4, 37.2, 36.9, 31.1, 25.8, 22.9, 22.1, 16.6.

HRMS (GC-EI) m/z calculated for C₁₂H₂₂O₃+ [M]⁺214.1563; found: 214.1559.

¹H and ¹³C NMR data for the minor diastereomer epi-4:

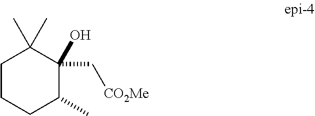

¹H NMR (CD₂Cl₂, 501 MHZ) δ (ppm)=4.63 (s, 1H), 3.68 (s, 3H), 2.56 (d, J=16.0 Hz, 1H), 2.48 (d, J=16.0 Hz, 1H), 1.95 (dqd, J=13.5, 6.8, 3.8 Hz, 1H), 1.60-1.56 (m, 1H), 1.55-1.48 (m, 1H), 1.46-1.42 (m, 1H), 1.37-1.32 (m, 2H), 1.07 (qd, J=13.2, 5.1 Hz, 1H), 1.01 (s, 3H), 0.86 (d, J=6.8 Hz, 3H), 0.86 (s, 3H).

¹³C NMR (CD₂Cl₂, 151 MHz) δ (ppm)=176.2, 77.1, 52.2, 40.1, 38.0, 37.8, 32.5, 31.9, 26.1, 22.2, 21.6, 16.2.

### 3. Olfactory Impression

In order to test the quality and intensity of the compounds of the present invention and the mixtures, scent strip tests were performed.

For this purpose, strips of absorbent paper were dipped into a solution containing 1 to 10 wt. % of the compound to be tested in triethyl citrate. After evaporation of the solvent (about 30 s) the scent impression was olfactorically evaluated by a trained perfumer.

The aroma impression of the prepared mixtures according to the present invention comprising compounds of formula (Ia) and compounds of formula (Ib) is indicated in the below Table I The compound was formulated in the perfume compositions according to Tables II and III and were labelled as compound A.

Compounds according to compound of formula (Ia), (lb) and (Ic) are:

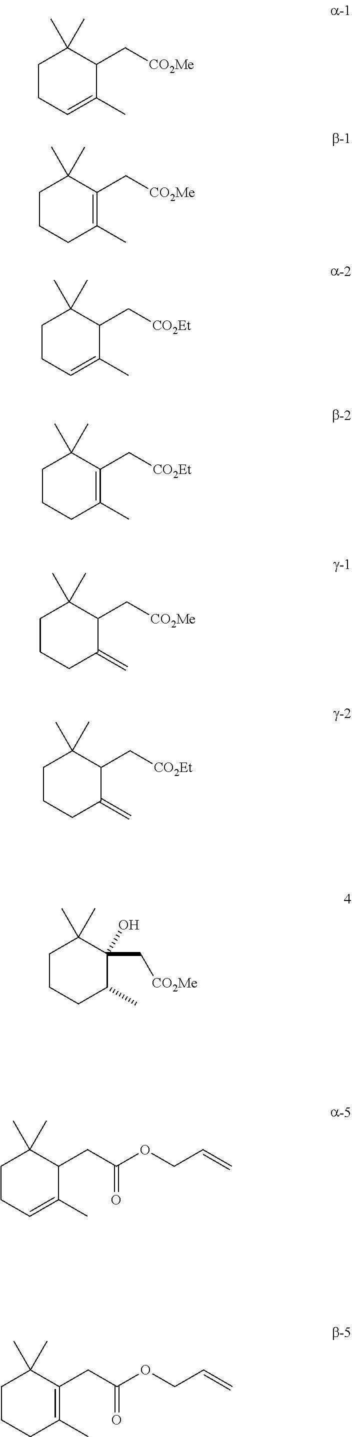

TABLE I
Aroma impressions of the mixtures

| | Ratio of the | |
|---|---|---|
| | mixtures (based on | |
| Sample | GC-%/NMR-%)/pure | |
| no. | compound | Aroma impression |
| S1 | α-1:β-1:γ-1 | Citrus, Petitgrain, Floral, |
| | 42.5:48.4:2.7 | Damascone, Tobacco |
| S2 | α-1:β-1:γ-1 | Similar to S1, but Citrus note |
| | 80.6:18.4:0.9 | is more enhanced |
| S3 | α-1:β-1:γ-1 | Similar to S1 but Tobacco note |
| | 0.9:91.8:4.5 | is more enhanced |
| S4 | α-2:β-2:γ-2 | Tobacco, Woody, Gourmand, |
| | 45:55:<1 | Sweet |
| S5 | α-2:β-2:γ-2 | Tobacco, Woody, Gourmand, |
| | 40:60:<1 | Sweet |
| S6 | α-2:β-2:γ-2 | Tobacco, Woody, Gourmand, |
| | 28:72:<1 | Sweet |
| S7 | α-1:β-1:γ-1 | Woody, Fruity, Vetiver, Green, |
| | 31:68:1 | Herbal, Citrus, Petitgrain, |
| | | Rooty |
| S8 | γ-1 | Damascone, Tobacco, Ionone, |
| | | Ambra |
| S9 | α-1 | Damascone, Petitgrain, Fresh |
| S10 | β-1 | Woody, Fruity, Vetiver, Green, |
| | | Herbal, Citrus, Petigrain, |
| | | Rooty |
| S11 | 3 | Leather and Plastic |
| S12 | α-5:β-5 = 55:45 | Green |
| S13 | 4 | Floral, Chrysanthemum, |
| | | Natural, Fir Needle, |
| | | Camphoraceous |

### 4. Advantageous Compositions

The mixtures/compounds as indicated in Table I were formulated as compositions according to tables II and III. Mixtures indicated in Table I were labelled as “compound A” in Table II and III.

TABLE II
Compositions 1A and 1B

| | 1A | 1B |
|---|---|---|
| Lactone C10 gamma (5-hexyloxolan-2-one) | 2 | 2 |
| Bourgeonal (3-(4-tert-butylphenyl)propanal) | 2 | 2 |
| Citronellol | 3 | 3 |
| Aldehyde C-14 (5-heptyloxolan-2-one) | 3 | 3 |
| Allyl heptylate | 4 | 4 |
| Amber core (1-(2-tert-butylcyclohexyl)oxybutan-2-ol) | 4 | 4 |
| Ethyl-2-methyl butyrate | 4 | 4 |
| Geranyl acetate | 5 | 5 |
| Helional (3-(1,3-benzodioxol-5-yl)-2-methylpropanal) | 10 | 10 |
| Manzanate (ethyl 2-methylpentanoate) | 10 | 10 |
| Amberwood (ethoxymethoxycyclododecane) | 10 | 10 |
| Hexyl acetate | 11 | 11 |
| Benzyl salicylate | 12 | 12 |
| Magnolan (2,4-dimethyl-4,4a,5,9b-tetrahydroindeno[1,2- | 15 | 15 |
| d][1,3]dioxine) | | |
| Verdox (2-tert-butylcyclohexyl) acetate) | 25 | 25 |
| Bergamot oil bergaptene free | 25 | 25 |
| Linalool | 30 | 30 |
| Dipropylene glycol | 45 | 45 |
| Iso E Super (Tetramethyl acetyloctahydronaphthalenes) | 110 | 110 |
| Pyranol (4-methyl-2-(2-methylpropyl)oxan-4-ol) | 170 | 170 |
| Hedione (methyl 3-oxo-2-pentylcyclopentaneacetate) | 200 | 200 |
| Galaxolide 50% IPM (1,3,4,6,7,8-hexahydro-4,6,6,7,8,8- | 300 | 300 |
| hexamethylcyclopenta(g)-2-benzopyran 50% in isopropyl | | |
| myristate) | | |
| compound A | 10 | 20 |
| Total (parts by weight) | 1020 | 1050 |

TABLE III
Compositions 2A and 2B

| | 2A | 2B |
|---|---|---|
| Raspberry ketone (4-(4-hydroxyphenyl)butan-2-one) | 4 | 4 |
| Vanitrope (2-ethoxy-5-prop-1-enylphenol) | 6 | 6 |
| Cyclamen aldehyde (at least 90% 2-methyl-3-(p- | 10 | 10 |
| isopropylphenyl)-propionaldehyde; secondary component: | | |
| 5% 3-(p-cumenyl)-2-methylpropionic acid) | | |
| Bicyclononalactone (3,4,4a,5,6,7,8,8a-octahydrochromen-2- | 10 | 10 |
| one) | | |
| Aldehyde C-14 (5-heptyloxolan-2-one) | 14 | 14 |
| Ethylvanillin (3-ethoxy-4-hydroxybenzaldehyde) | 16 | 16 |
| Heliotropine (1,3-benzodioxole-5-carbaldehyde) | 20 | 20 |
| Iso E Super (tetramethyl acetyloctahydronaphthalenes) | 20 | 20 |
| Sandela (3-[5,5,6-trimethylbicyclo[2.2.1]hept-2- | 30 | 30 |
| yl]cyclohexan-1-ol) | | |
| Vanillin isobutyrate ((4-formyl-2-methoxyphenyl) 2- | 40 | 40 |
| methylpropanoate) | | |
| Aldehyde C-18 (5-pentyloxolan-2-one) | 50 | 50 |
| Benzyl salicylate | 60 | 60 |
| Hexyl cinnamic aldehyde (2-(phenylmethylidene)octanal) | 70 | 70 |
| Hedione (methyl 3-oxo-2-pentylcyclopentaneacetate) | 130 | 130 |
| Pyranol (4-methyl-2-(2-methylpropyl)oxan-4-ol) | 150 | 150 |
| Ethylene brassylate (1,4-dioxacycloheptadecane-5,17-dione) | 170 | 170 |
| Galaxolide 50% IPM (1,3,4,6,7,8-hexahydro-4,6,6,7,8,8- | 200 | 200 |
| hexamethylcyclopenta(g)-2-benzopyran 50% in isopropyl | | |
| myristate) | | |
| compound A | 10 | 20 |
| Total (parts by weight) | 1020 | 1050 |

Composition according to table II and table III namely 1A, 1B, 2A,2B could be included in various compositions selected from the group consisting of Deo pump spray, Clean hair-conditioner, Face wash gel, Foam bath concentrate, Hair gel, Self-foaming bodywash, Sprayable sun care emulsion, Sprayable sun protection emulsion, Emollient facial gel, 2-phases oil foam bath, Shampoos, Shower bath, Hydro-alcoholic AP/Deo pump spray, Aerosol, Aqueous/alcoholic AP/Deo roll-on, Styling Gel Type “Out of Bed”, Shaving Foam, Sensitive skin Baby shampoo, Body wash for Sensitive Skin, Gloss Enhancing Shampoo for Sensitive Scalp, Deo Stick, Baby 10

Wipe, After shave balm, Face Gel, Face Day Care Cream, Face Cleanser, Body lotion, Sun Care SPF50+, Sprayable Lotion, Hand dish cleaner-regular, Hand dish cleaner-concentrate, Sanitary cleaner-concentrate, All-purpose cleaner, Anti-bacterial fabric softener, Detergent composition, Powder detergent composition and Liquid detergent composition.

A person skilled in art may be well versed with the various general formulations for the above-mentioned products.

Compositions 1A, 1B, 2A and 2B can for example be formulated in specific formulations as disclosed in IP.com Number: IPCOM000258614D entitled New Aroma Chemicals pages 6 to 46, Table 1 to Table D13, wherein the “Fragrance Composition 1A” is replaced by identical amounts of compositions 1A, 1B, 2A or 2B.

1-16. (canceled)

17. A compound of formula (I) 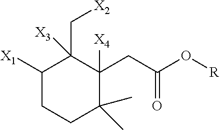
compound of formula (I)
or stereoisomers thereof,
where,
X₁ and X₃ together form a double bond between the carbon atoms to which they are bound, with the proviso that X₂ and X₄ are hydrogen; or
X₃ and X₄ together form a double bond between the carbon atoms to which they are bound, with the proviso that X₁ and X₂ are hydrogen; or
X₂ and X₃ together form a double bond between the carbon atoms to which they are bound, with the proviso that X₁ and X₄ are hydrogen, or
X₄ is OH, X₁, X₂ and X₃ are all hydrogen
where R is selected from C₁-C₅ linear or branched alkyl and C₃-C₅ linear or branched alkenyl.

18. The compound according to claim 17, wherein compound of formula (I) comprises, at least one compound of formula (Ia) 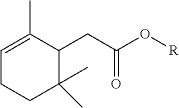
compound of formula (Ia)
or its stereoisomers or mix of its stereoisomers,
or at least one compound of formula (Ib), 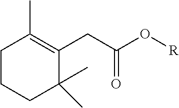
compound of formula (Ib),
or at least one compound of formula (Ic), 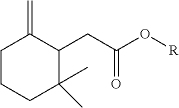
compound of formula (Ic)
or its stereoisomers or mix of its stereoisomers,
or at least one compound of formula (Id) 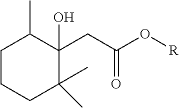
compound of formula (Id)
or its stereoisomers or mix of its stereoisomers,
or mixtures thereof,
where,
R is selected from C₁-C₅ linear or branched alkyl and C₃-C₅ linear or branched alkenyl.

19. The compound according to claim 17, wherein R is selected from methyl, ethyl, propyl, butyl, isobutyl, isopropyl, 1-propenyl, or 2-propenyl.

20. The compound according to claim 17, wherein R is selected from methyl ethyl or 1-propenyl.

21. The compound according to claim 18, wherein the weight ratio of the at least one compound of formula (Ia) and at least one compound of formula (Ib) is in the range of 0.1:99.9 to 99.9:0.1.

22. The compound according to claim 18, wherein the weight ratio of the at least one compound of formula (Ia) and at least one compound of formula (Ib) is in the range of 40:60 to 60:40.

23. The compound according to claim 18, wherein the mixture has a ratio in the range of 90:10 to 99.9:0.1, wherein the ratio represents the combined amount of compound of formula (Ia) and compound of formula (Ib) to the compound of formula (Ic).

24. A method of imparting an aroma impression to a composition, comprising at least the step of adding the compound of formula (I) according to claim 17 to a composition.

25. The compound according to claim 17, wherein the aroma impression is selected from the group consisting of woody note, fruity note, herbal note, green note, rooty note, citrus note, petitgrain note, floral note, damascone note, tobacco note, ashtray note, gourmand note, sweet note, vetiver note, ionone note, ambra note, fresh note, natural note, fir needle note, camphoraceous note, or any combination of two or more of these.

26. The compound according to claim 17, wherein the at least one compound of formula (I) is used in a total amount in the range of >0.001 wt. % to ≤70.0 wt. %, based on the total weight of the composition.

27. A composition comprising,
i) at least one compound of formula (I) according to claim 17, and
ji) at least one aroma chemical (X) other than compounds of formula (1), or
iii) at least one non-aroma chemical carrier, or
iv) both of (ii) and (iii).

28. The composition according to claim 27, wherein the at least one compound of formula (I) is present in the range of >0.01 wt. % to ≤70.0 wt. %, based on the total weight of the composition.

29. The composition according to claim 27, wherein the at least one non-aroma chemical carrier (ii) is selected from surfactants, oil components, antioxidants, deodorant-active agents, or solvents.

30. The composition according to claim 27, wherein the composition is selected from perfume compositions, body care compositions, hygiene articles, cleaning compositions, textile detergent compositions, compositions for scent dispensers, foods, food supplements, pharmaceutical compositions, or crop protection compositions.

31. A mixture comprising at least one compound of formula (Ib), 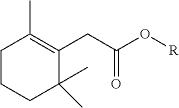
compound of formula (Ib),
or its stereoisomers or mix of its stereoisomers,
and
at least one compound of formula (IV) 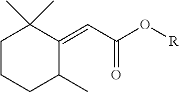
compound of formula (IV)
or its stereoisomers or mix of its stereoisomers where,
R is selected from C₁-C₅ linear or branched alkyl and C₃-C₅ linear or branched alkenyl.

32. A process for preparing the mixture of at least one compound of formula (I) according to claim 17, comprising at least the step of
a) Cyclization of the compound of formula (III) 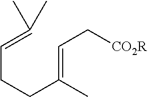
compound of formula (III)
where R is selected from C₁-C₅ linear or branched alkyl and C₃-C₅ linear or branched alkenyl, using catalytic amount of acid to produce the at least one compound of formula (I),
b) optionally purifying the at least one compound of formula (I).
